(19) United States
(12) Patent Application Publication
(10) Pub. No.: US 2013/0273546 A1
(43) Pub. Date: Oct. 17, 2013

(54) LOW TEMPERATURE ENZYME AND METHOD THEREOF

(71) Applicant: Universiti Putra Malaysia, (US)

(72) Inventors: Raja Noor Zaliha, Serdang Baharu (MY); Abu Bakar Salleh, Selangor (MY); Mahiran Basri, Selangor (MY); Norsyuhada Alias, Selangor (MY)

(73) Assignee: UNIVERSITI PUTRA MALAYSIA

(21) Appl. No.: 13/850,862

(22) Filed: Mar. 26, 2013

Related U.S. Application Data
(63) Continuation of application No. 12/587,869, filed on Oct. 13, 2009, now abandoned.

(30) Foreign Application Priority Data
Feb. 25, 2009 (MY) 20090756

(51) Int. Cl. C12N 9/58; C12Q 1/68

(52) U.S. Cl. CPC C12N 9/58; C12Q 1/6895
USPC 435/6.12; 435/223; 536/23.2; 435/6.18

(57) ABSTRACT

The present invention discloses manufacturing method of low temperature protease and special yeast strain, which can generate low temperature protease in the condition of low temperatures. More particularly, the present invention is to obtain a protease (preferably Cold-adapted protease PI12) using the marine strain of the Leucosporidium antarcticum sp. (NCYC accession no: 3391) for possible use in industries.

### FIELD OF INVENTION

The present invention also opens an alternate use of low-temperature yeast methods and by the method of low-temperature protease products. The low-temperature protease can be used as the main enzyme detergent additives can be directly room temperature washing, saves energy; and in food manufacturing, leather, silk, environmental protection, medicine and other fields has broad application prospects.

### BACKGROUND OF INVENTION

Enzymes are established active ingredients of wash and detergents, which serve in particular for the cleaning of hard surfaces or from textile ones. Proteases are involved in a wide variety of biological processes. Cold-adapted enzymes are enzymes that derived from obligate psychrophilic microorganisms that have the capability to catalyze chemical reactions at low temperature. This is due to the development of adaptation strategies of psychrophilic organisms that lived in permanently cold habitats such as, the development of adaptation in the form of finely tuned structural changes in their membranes, constitutive proteins and enzymes as well as molecular adjustments which enabling them to compensate for the deleterious effects of low temperature. Enzymes from psychrophiles are essentially alike their counterparts of meso- and thermophilic origin wherein they have the same overall fold and they catalyze identical reactions in the same way.

Proteases are degradative enzymes that split up proteins into their component amino acids. The process is called peptide cleavage, a common mechanism of activation or inactivation of enzymes. Proteases conduct highly specific and selective modifications of proteins such as activation of zymogenic forms of enzymes by limited proteolysis, blood clotting and lysis of fibrin clots, processing and transport of secretory proteins across the membrane. Proteases execute a large variety of functions and take part in numerous biochemical reactions in living organisms including formation of spore and germination, coagulation, cascade reactions, post translation reactions, modulation of gene expression, enzyme modification and secretion of various protein enzymes biocatalyst.

Disruption of the balance between proteases and protease inhibitors is often associated with pathologic tissue destruction. Various studies have focused on the role of proteinases in tissue injury, and it is thought that the balance between proteinases and proteinase inhibitors is a major determinant in maintaining tissue integrity.

The use of proteases in wash and detergents becomes for example in the Patent Laid opens WHERE 93/07276 and WHERE 96/28566 described. Attempts to use fungi or protease systems from fungi and bacteria for various applications as mentioned above are reported in literature. Microbial alkaline proteases (subtilisins: E.C. 3.4.21.14) are a commercially important group of enzymes for detergent industries. Ideally, proteases used in detergent industries should have high activity and stability over a broad range of pH and temperature

This invention discloses a manufacturing method of low temperature protease and special yeast strain, with the goal of providing a kind of yeast which can generate low temperature protease in the condition of low temperatures. The protease produced by this invention has low temperature, good stability and is applicable in industry of abluent, feedstuff, leather and food handling.

### SUMMARY OF INVENTION

Accordingly, the present invention relates to a novel enzyme obtained from a biologically pure strain, the strain is isolated from antarctic marine waters, wherein the biologically pure strain is Leucosporidium antarcticum sp. The pure strain having the capability to grow between 4° C. and 20° C. and the Leucosporidium antarcticum sp (preferably known as Leucosporidium antarcticum strain PI12) have been deposited in the National Collection of Yeast Cultures (NCYC) under the NCYC number 3391. Moreover, the Leucosporidium antarcticum sp or Leucosporidium antarcticum strain PI12 having the capability to produce a protein and indeed, the enzyme comprising a nucleotide sequence of SEQ ID NO: 1 comprising an amino acid sequence of SEQ ID NO: 2. The novel enzyme is a known to produce protease preferably a cold active protease PI12. In addition, the present invention also relates to a method of indentifying the Leucosporidium antarcticum sp, whereby the method includes isolating the Leucosporidium antarcticum PI12 in a solid media and antibiotics for at least 10 days at 4° C. and characterizing the Leucosporidium antarcticum PI12 as psychrophilic isolate PI12, identifying the morphology and size of the psychrophilic isolate, scanning the psychrophilic isolate under an electron microscopy and transmission microscopy, conducting a ribosomal RNA identification by using 16srRNA, 18rRNA and ITS1/ITS2 primers, amplifying the primers by performing PCR, obtaining amplicons and examining the amplicons, purifying and sequencing the amplicons and obtaining sequences from the above primers. Interestingly, the Leucosporidium antarcticum sp is resistant to ampicillin, streptamycin and chloramphenicol and it is said that the Leucosporidium antarcticum sp comprising a nucleotide sequence of SEQ ID NO 3 and SEQ ID NO 4.

More particularly, the present invention relates to a gene coding a protein from the cold active protease PI12 enzyme and the protein having a size of about 99.3 kDa.

Furthermore, the present invention also relates to a method of obtaining a PI12 protease gene isolated from Leucosporidium antarcticum sp, wherein the method includes; conducting DNA extraction of the Leucosporidium antarcticum sp, obtaining a purified DNA extract, identifying the partial putative protease gene in a recombinant plasmid by performing double digestion using EcoRI restriction enzyme at 37° C. for 1 hour and terminated at 65° C. for 20 minutes, obtaining a digested product and sequencing the digested product, conducting RNA extraction of the Leucosporidium antarcticum sp, performing RT-PCR to amplify the protease gene, performing an amplification of cDNA ends and obtaining PCR product, cloning and sequencing the PCR product, obtaining a full length sequence of a mature PI12 protease gene. The PI12 protease gene is amplified at 2892 bp and encodes for 963 amino acids. This method further includes cloning of the mature PI12 protease gene into an expression vector (Pichia pastoris (pPIC9) and obtaining low temperature or cold-adapted PI12 protease clones at 15° C. for about 30 minutes. The clones obtained includes GS115 strain and KM71 strain, wherein GS115 strain is GpPro1 and GpPro2 and the KM71 strain is KpPro1. The clones were further verified by assaying with azocasein as a substrate and terminated using trichloroacetic acid.

### BRIEF DESCRIPTION OF THE DRAWINGS

FIG. 1 shows Casein hydrolysis on Skim Milk Agar Plate.

FIG. 2 shows Antarctic Microorganism Strain PI12.

FIG. 3 shows Scanning electron microscopy (SEM) of Antarctic Microorganism Strain PI12.

FIG. 4 shows Transmission electron microscopy (TEM) of Antarctic Microorganism Strain PI12.

FIG. 5 shows Ribosomal DNA (rDNA) organization.

FIG. 6 shows PCR products of ITS1/ITS2 and 18S rDNA amplicon.

FIG. 7 shows 18S rDNA sequence of Leucosporidium antarcticum strain PI12

FIG. 8 shows ITS1/5.8S rDNA/ITS2 sequence of Leucosporidium antarcticum strain PI12.

FIG. 9 shows Neighbor-joining Phylogenetic Analysis of Internal Transcribed Spacer 1 (ITS1)/5.88 rRNA Gene/Internal Transcribed Spacer 2 (ITS2).

FIG. 10 shows the PCR products of DNA walking.

FIG. 11 shows Sequence alignment of PI12 protease gene from DNA walking.

FIG. 12 shows PCR products of putative protease gene of Leucosporidium antarcticum strain PI12.

FIG. 13 shows Agarose gel of RACE PCR product.

FIG. 14 shows full-length cDNA of PI12 protease gene.

FIG. 15 shows Nucleotide and deduced amino acid sequences of genomic DNA and cDNAs encoding protease from Leucosporidium antarcticum strain PI12.

FIG. 16 shows Analysis of recombinant plasmid, pPIC9.

FIG. 17 shows Gel electrophoresis of linearized plasmid.

FIG. 18 shows PCR products from positive colonies of recombinant Pichia pastoris (P. pastoris).

FIG. 19 shows Secretion of PI12 protease activity by P. pastoris GS 115 and KM71 clones.

FIG. 20 shows SDS-PAGE expression of PI12 protease from different clones.

### DETAILED DESCRIPTION

The present invention relates to a process for production of protease enzyme, using a marine water strain of Leucosporidium antarcticum sp., isolated from Antarctic marine water and deposited in the National Collection of Yeast Cultures (NCYC) under the NCYC number 3391. Furthermore, the protease produced by the said Leucosporidium antarcticum or also known as Leucosporidium antarcticum strain PI12 (NCYC accession no: 3391) is active at and temperature of 4 [deg.] C. to 20[deg.] C. and optimum at 4[deg.] C. One hundred percent of the enzyme activity is retained after incubating the enzyme at 4[deg.] C. for about 10 days. The main object of the present invention is to isolate a novel marine strain of the Leucosporidium antarcticum sp., from marine water. Another main objective of the present invention is to provide a process for production of protease (preferably Cold-adapted protease PI12) using the marine strain of the Leucosporidium antarcticum sp. (NCYC accession no: 3391) for possible use in industries wherever protease is required which obviates drawbacks as detailed above.

The Cold-adapted protease PI12 is derived from obligate psychrophilic yeast that has the capability to catalyze chemical reactions at low temperature. More particularly cold-active enzyme might offer novel opportunities for biotechnological exploitation based on their high catalytic activity at low temperatures, low thermostability and unusual specificities. The PI12 or psychrophilic isolate was identified as basidiomycete yeast, Leucosporidium antarcticum via 18S rDNA, 26S rDNA and ITS1/ITS2 gene sequence. The 18S rDNA and ITS1/5.8S rDNA/ITS2 sequence of psychrophilic yeast Leucosporidium antarcticum strain PI12 have been deposited into GenBank data library with the accession number EU621372 and FJ554838, respectively. This is a aerobic budding yeast can grow only below 15° C.

Sequence Listing

| SEQ ID NO: 1 | |
|---|---|
| ATGCTCTTCCTCCCCGTCCTCCTCCTCCTCCTTCCCGGCGTCACTGCCTT | |
| CCTCAACCCAGTTACCAACCGCGCGACCAACGCCATCTCCTCGACTCAAT | |
| ACCTCTCTAACGCCTATATCCTCGAATTGGACCTCTCCACCCCCGGCCTC | |
| GTCAAACGGGATAGCACGCCCGACTCTATCCTAGAGGACGTACTCACGTC | |
| CGTAGGCCGCAACGGTATCAAGTACCAACTCCGCCACCGCTTTATCTCCC | |
| CGACTCTGTTCCACGGCGCTTCGATCACTGTCCCCCCTGGAATCTCCCGC | |
| TCCCAAATCGCCTCTCTCCGCGGTATCAAACGCGTCTGGCCCGTTCGAAA | |
| GTTCTCCCGACCCAGCGCAGTAGTGGACGCCGATGGAGGAGGAAGCGGGT | |
| TCTCAGGGTCGCCTATCAAGGCGGCGCTCATGGGGGTGAAAGAGCTCGGG | |
| AAGCGCGCGAACGCTTATGCTGGAGATACGTTTGGACCGCATGTCATGAC | |
| GGGGGTTAATGAGACGCATGAGGCGGGGTTGTTGGGAGCTGGGATTAAGA | |
| TTGGGGTGCTGGACACTGGTGTTGATTATTTGAACCCGATTCTGGGAGGC | |
| TGCTTTGGACCTGGGTGCCATATGTCGTTTGGGTACGACTTGGTTGGCGA | |
| TGATTACGATGGAGATAACGCTCCTGTGCCGGATGTGGATCCTTTTGCGA | |
| GCTGCGATCCTCATGGAACTCACGTTACGGGAATCATTGGAGCGCTCCCG | |
| AATGCGTTTGGATTTACTGGCGTCGCACCCGCCGCTACTCTGGGCCACTA | |
| CCGAGTATTTGGCTGCACTGGCTTCGTCGGAGAAGATATCATTCTCGCTG | |
| GACTCATGCGAGGAGTCGAGGACAACTGCAACGTCTTGACCCTCTCTCTC | |
| GGAGGTCCAGGAGGGTGGGTCAAGGGCACGCCGGCGTCCATCCTTATCGA | |
| CCAGATCGAAGCGCAAGGCATTCTCGTCACCGTCGCCACTGGCAACTCGG | |
| GAGCTGAGGGCATGTTCTTCTCCGAGTCTCCCGCCTCGACGATCAACGGC | |
| CTTTCCATCGCATCCACGGACGTTACCGACCTCATCGCCTACAACGCCAC | |
| CGTCTCAGGCCAACCTGCGATCCCTTACCTCTCCGCCACGCCCCTCAACG | |
| TCGTCGCCAACAGCTTCCGCGTCCACTTCACCTCTACCGACCCCAACAAC | |
| CCCGTCGACGCCTGCTCTCCTCTTCCGGCTGGAGCGCCCGACTTCGCCAA | |
| CTATGTTACGGTCGTTCAGCGTGGGACTTGTACGTTCGTTACCAAGTACC | |
| AGAACGTTCTCAATGCTGGAGGAAAGATCGTATTGTTGTACAACTCGGAG | |
| GGAGCTGGGAACCTCCCTTACCTCACGCCCAACGGTGTCGGCATCGACGC | |
| CGTTGCAGGTCTTCGTCGTTCCGACGGACTCAAGCTTCTCTCGTACTATC | |
| AGAATGCCAACAAGCGTCTCACTCTGCGCTTCCCCAAGGGCAAGATCGTC | |
| GCAGGCTTGACCGATACCATCACCGGCGGACTCATCTCGGGTTACTCGAC | |
| GTTTGGTCCGACGAATGACCTCTACGGTCAGCCTACCCTCTCTGCCCCTG | |
| GTGGCAACATCCTTTCGACCTTCCCTCTCTCCGAGGGAGGAGTGGCGOTC | |
| ATCAGTGGGACGAGCATGTCGTGCCCCTTTGTCGCTGGATCTGCGGCGGT | |
| CCTCATGGCCGCTCGCGCTTCGGAGAACCTCACGCCGCTTGAGATCAGGA | |
| GTCTCCTTACTACCACTGCGAAGCTTACGCCGGTCTCGCTCTTGGGATCG | |
| ACGCCTTTGGTGAGCGTGATTCGTCAAGGAGGAGGACTCGTTCAGGTTGC | |
| CAAGGCGCTCGCGGCCAAGACGCTAATCTCTCCTCACGAGCTCCTGCTCA | |
| ACGACACTGCGAACGCGAACTACGTCCAGACTATCAAGATCAAGAACACC | |
| AACTCGTGGGCGATGAAGTACACCTTCTCCTCGGCCGTCGCCCAAGGACT | |
| CGGAACTTTCGACGCTTCGGGCGATATCCTCCCTACCCTCGACCCAGTCG | |
| CCGTCTCTGGCGCACAGGCTACCGTCGCGTTCAACACTCGGATCCTCAGC | |
| GTCGCGCCCGGCGCGACGGGGTCCGTCGTGGCGACTATCACGCCGCCGGT | |
| TCTTCCCGTAGCGGACGCTGCGAGGTTCCCTATCTTCTCTGGGTGGATCA | |
| GGGTGAATGGGCAAGGCGCGAGGGATAGCAGTAGGAACGAGGCGTACACT | |
| GTCCCGTACTTTGGGCTTGCGGCGAAGATGATCGATATGCAAGTCCTCGA | |
| CACCACCGAGACCATTTACGGTCCGGGCTACGCCTACCCCTTCGTGATCG | |
| ACGACGCGATTGGAGACATCCAATCCACCACAACGTCGTACTCCAGGAAC | |
| CTCOGGCCCACCGTCTTCGCTCGCTTTGCCACTGGAACCCTTCACTACAG | |
| CCTCGATCTCGTCCTAGCCGACATCGCCTTTACCCCCACCTACCCCAACT | |
| CCTCCCCCGCCACTCGTTTCGTCAAGCGCTCCCTCACGCAGCACACCTCT | |
| GCCGCTTCGCACCTCGCCAAGCGCCGCGTCTCCATCGCCACTATCAACCC | |
| CAAAGCCACCCTCGTCGCCGATCGACAGCTCCACTCGGACGTCCCTATCG | |
| AGGGCAACATCTTCACCCAACCCTTTACTGGAAGGGATTACCTCGTCGAC | |
| GCAGCCCCGACGGGATCCACCGATCGTACCGTCACTTTTAACGGGCAGTA | |
| CGCCGAGAACGGCCTCGTGAGGACGGCTGTGACGGGGACTTCGTACCGCT | |
| TCCTCCTTCGGGCGTTGAAGATCTCGGGAGACGCGATGTACGAGGATCAG | |
| TATGAGAGCTGGCTCTCGCTACCGTTCTCGTTCCGTGCGTAG | |
| SEQ ID NO: 2 | |
| MLFLPVLLLLLPGVTAFLNPVTNRATNAISSTQYLSNAYILELDLSTPGLVKRDSTPDSILEDVLTSVGR | |
| NGIKYQLRHRFISPTLFHGASITVPPGISRSQIASLRGIKRVWPVRKFSRPSAVVDADGGGSGFSGSPIK | |
| AALMGVKELGKRANAYAGDTFGPHVMTGVNETHEAGLLGAGIKIGVLDTGVDYLNPILGGCFGPGCHMSF | |
| GYDLVGDDYDGDNAPVPDVDPFASCDPHGTHVTGIIGALPNAFGFTGVAPAATLGHYRVFGCTGFVGEDI | |
| ILAGLMRGVEDNCNVLTLSLGGPGGWVKGTPASILIDQIEAQGILVTVATGNSGAEGMFFSESPASTING | |
| LSIASTAVTDLIAYNATVSGQPAIPYLSATPLNVVANSFRVHFTSTDPNNPVDACSPLPAGAPDFANYVT | |
| VVQRGTCTFVTKYQNVLNAGGKIVLLYNSEGAGNLPYLTPNGVGIDAVAGLRRSDGLKLLSYYQNANKRL | |
| TLRFPKGKIVAGLTDTITGGLISGYSTFGPTNDLYGQPTLSAPGGNILSTFPLSEGGVAVISGTSMSCPF | |
| VAGSAAVLMAARASENLTPLEIRSLLTTTAKLTPVSLLGSTPLVSVIRQGGGLVQVAKALAAKTLISPHE | |
| LLLNDTANANYVQTIKIKNTNSWAMKYTFSSAVAQGLGTFDASGDILPTLDPVAVSGAQATVAFNTRILS | |
| VAPGATGSVVATITPPVLPVADAARFPIFSGWIRVNGQGARDSSRNEAYTVPYFGLAAKMIDMQVLDTTE | |
| TIYGPGYAYPFVIDDAIGDIQSTTTSYSRNLGPTVFARFATGTLHYSLDLVLADIAFTPTYPNSSPATRF | |
| VKRSLTQHTSAASHLAKRRVSIATINPKATLVADRQLHSDVPIEGNIFTQPFTGRDYLVDAAPTGSTDRT | |
| VTFNGQYAENGLVRTAVTGTSYRFLLRALKISGDAMYEDQYESWLSLPFSFRA | |
| SEQ ID NO: 3 | |
| GCTTGTCTCAAGATTAAGCCATGCATGTCTAAGTTTAAGCAATAAACGGTGAAACTGCGA | 60 |
| ATGGCTCATTAAATCAGTCATAGTTTATTTGATGGTACCCTACTACATGGATAACTGTGG | 120 |
| TAATTCTAGAGCTAATACATGCCGAAAAATCTCGACTTCTGGAAGAGATGTATTTATTAG | 180 |
| ATCCAAAACCAGTGGCCTTCGGGTCTCCTTGGTGAATCATGATAACTGCTCGAATCGCAT | 240 |
| GGCCTTGCGCCGGCGATGCTTCATTCAAATATCTGCCCTATCAACTTTCGATGGTAGGAT | 300 |
| AGAGGCCTACCATGGTGATGACGGGTAACGOGGAATAAGGGTTCGATTCCGGAGAGAGGG | 360 |
| CCTGAGAAACGGCCCTCAGGTCTAAGGACACGCAGCAGGCGCGCAAATTATCCCCTGGCA | 420 |
| ACACTTTGCCGAGATAGTGACAATAAATAACAATGCAGGGCTCTTACGGGTCTTGCAATT | 480 |
| GGAATGAGTACAATTTAAATCCCTTAACGAGGATCCATTGGAGGGCAAGTCTGGTGCCAG | 540 |
| CAGCCGCGGTAATTCCAGCTCCAATAGCGTATATTAAAGTTGTTGCCGTTAAAAAGCTCG | 600 |
| TAGTCGAACTTCGGTCCTTGTTGGTCGGTCCGCCTTCTTGGTGTGTACTTACTCAACGAG | 660 |
| GACTTACCTCCTGGTGAGCTGCAATGTCCTTTACTGGGTGTTGTAGGGAACCAGGACGTT | 720 |
| TACTTTGAAAAATTAGAGTGTTCAAAGCAGGCCTACGCCCGAATACATTAGCATGGAATA | 780 |
| TAGAATAGGACGCGCGTTCCCATTTTGTTGGTTTCTGAGATCGCCGTAATGATTAATAGG | 840 |
| GATAGTTGGGGGCATTCGTATTCCGTCGTCAGAGGTGAAATTCTTGGATTGCCGGAAGAC | 900 |
| GAACTATTGCGAAAGCATTTGCCAAGGATGTTTTCATTGATCAAGAACGAAGGAAGGGGG | 960 |
| ATCGAAAACGATCAGATACCGTTGTTGTCTCTTCTGTAAACTATGCCAATTGGGGATTAG | 1020 |
| CTCAGGATTTTTAATGACTGAGTTAGCACCCGAAGAGAAATCTTTAAATGAGGTTCGGGG | 1080 |
| GGGAGTATGGTCGCAAGGCTGAAACTTAAAGGAATTGACGGAAGGGCACCACCAGGTGTG | 1140 |
| GAGCCTGCGGCTTAATTTGACTCAACACGGGGAAACTCACCAGGTCCAGACACAATAAGG | 1200 |
| ATTGACAGATTGATAGCTCTTTCTTGATCTTGTGGTTGGTGGTGCATGGCCGTTCTTAGT | 1260 |
| TGGTGGAGTGATTTGTCTGGTTAATTCCGATAACGAACGAGACCTTAACCTGCTAAATAG | 1320 |
| ACCAGCCGGCTTTGGCTGGCTGCTGTCTTCTTAGAGGGACTATCAGCGTTTAGCTGATGG | 1380 |
| AAGTTTGAGGCAATAACAGGTCTGTGATGCCCTTAGATGTTCTGGGCCGCACGCGCGCTA | 1440 |
| CACTGACAGAGCCAGCGAGTCTACCACCTTGGCCGGAAGGCCTGGGTAATCTTGTGAAAC | 1500 |
| TCTGTCGTGATGGGGATAGAACATTGCAATTATTGTTCTTCAACGAGGAATACCTAGTAA | 1560 |
| GCGTGAGTCATCAGCTCGCGTTGATTACGTCCCTGCCCTTTGTACACACCGCCCGTCGCT | 1620 |
| ACTACCGATTGAATGGCTTAGTGAGGCCTCCAGATTGGCTATTAGGATCTCGCGAGAGAA | 1681 |
| CTTGACTGCTGAAAAGTTGTACGAACTTGGTCATTTAGAGGAAGTAAAAGTCGTAACAA | 1739 |
| SEQ ID NO: 4 | |
| TGGGGAAGGATCATTAGCGAATTTAGCGTTTCTCTTAACAGAGCGCGACCCTCCACTTTC | 60 |
| TTAACTCTGTGAACTTTTTTGGTCAAGCATGGCGTTTTCTCGATTGACTTTTATTAGAAA | 120 |
| GTTGGGACTACACATTTTGCTTGACGGCTCATTTTAAACACTAGTACAAGTATGTAACGA | 180 |
| AATATCGAAATATAAAAAAACTTTCAACAACGGATCTCTTGGCTCTCGCATCGATGAAGA | 240 |
| ACGCAGCGAAATGTGATAAGTAATGTGAATTGCAGAATTCAGTGAATCATCGAATCTTTG | 300 |
| AACGCACCTTGCGCTCCCTAGTATTCTGGGGAGCATGTCTGTTTGAGTGTCATGAACTCT | 360 |
| TCAACTCTATCGTTTCTTGTTAAGCGATTAGAAGTTTGGATTTTGAATGTTGCTAGTCCT | 420 |
| TTTACGGGACTTTAGCTCGTTCGTAATACATTAGCCTTTCTAATTCCGAACTTCGGATTG | 480 |
| ACTCAGTGTAATAGACTATTCGCTGAGGACACTAGTAATAGTGGCCGATATTCAACATAA | 540 |
| GAAAAGCTTCAAACCTTTGTAGTCAATTTTAGATTAGACCTCAGATCAGGCAGGATTAAC | 600 |
| CCGCCGAA | 608 |

### BEST MODE TO CARRY OUT THE INVENTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims

For the person skilled in the art obvious is, as it on the basis of nucleic acids in accordance with SEQ ID NO: 1 or polypeptides in accordance with SEQ ID NO: 2 nucleic acids and polypeptides of similar structure and more same or similar function obtained knows. As modification possibilities in particular those are the person skilled in the art prior art methods, fragmentation, deletion, insertion and fusion with other proteins or protein parts of mentioned. An optimization can take place for example in the adaptation to temperature influences, pH value variations or redox ratios. Desired ones are for example an improvement of the oxidation resistance, the stability opposite denaturing agents or proteolytic degradation, in relation to high temperatures, acidic or strong alkaline conditions, a change of the sensitivity opposite calcium ions or other CO factors, a reduction of the immunogenicity or all genes the effect. The other it can be desirable to change the surface charges and/or the isoelectric point of the polypeptides to optimize in particular in order the interactions with the substrate. An increase of the stability of the polypeptide can take place apart from a coupling to other peptides about also via coupling to polymers.

Other subject matter of the instant invention is therefore a Polynuecleotide selected from the group existing out:

a) Polynucleotide with a nucleic acid sequence in accordance with SEQ ID NO: 1,

b) Polynucleotide coding for a polypeptide with an amino acid sequence in accordance with SEQ ID NO: 2.

Furthermore subject matter of the instant invention is newer the use of the nucleic acids according to invention, to the production or isolation of nucleic acids according to invention, on the other hand to the production or isolation, to which nucleic acids according to invention homologous nucleic acids, in particular such also regarding the nucleic acids according to invention similar structural and same, similar and/or improved functional properties, whereby bottom functional properties in particular hydrolase activity, preferred proteolytic activity, and bottom improved functional properties for example higher specificity and/or higher conversion and/or higher stability of the enzyme with the desired reaction conditions, in particular regarding the pH value, which temperature is to be understood.

The nucleic acids according to invention know so for example as probes to the identification and/or isolation of homologous nucleic acids from a DNA, one cDNA or genomic gene bank, here preferably to the identification of nucleic acids, which here in particular become coding for hydrolases, in particular proteases, and/or for parts of it to code, or as anti scythe nucleic acids or as primers in the polymerase chain reaction (PCR), the amplification of nucleic acids comprising nucleic acids for hydrolases, preferred proteases, or parts of it, used in “expression cassette” or “expression vector” is a nucleic acid construct generated recombinantly or synthetically, with a series of specified nucleic acid elements that permit transcription of a particular nucleic acid in a target cell. The recombinant expression vector can be incorporated into a plasmid, chromosome, mitochondrial DNA, plastid DNA, virus, or nucleic acid fragment. Typically, the expression vector includes, among other sequences, a nucleic acid sequence to be transcribed and a promoter. Expression cassette may be used interchangeably with DNA construct and its grammatical equivalents. As used herein, the term “vector” refers to a nucleic acid construct designed to transfer nucleic acid sequences into cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art. As used herein, the term “plasmid” refers to a circular double-stranded (ds) DNA construct used as a cloning vector, and which forms an extrachromosomal self-replicating genetic element in some eukaryotes or integrates into the host chromosomes. The term “nucleic acid molecule” or “nucleic acid sequence” includes RNA, DNA and cDNA molecules. It will be understood that, as a result of the degeneracy of the genetic code, a multitude of nucleotide sequences encoding a given protein may be produced.

Other subject matter of the instant invention is therefore likewise a method to the isolation, preferred protease, or for parts of it coding nucleic acid, subsequent comprising the step steps of:

a) on the basis of a nucleic acid according to invention primers is prepared,

b) the primers in accordance with (a) become used, in order to be amplified in the PCR nucleic acids, above all cDNAs, unknown nucleic acid sequence,

c) the nucleic acids obtained in accordance with (b) by amplification are sequenced. Other subject matter of the instant invention is a method to the production of a nucleic acid according to invention, characterised in that the nucleic acid chemical synthesized becomes. The nucleic acids according to invention can do for example chemical on the basis in SEQ ID NO: 1 indicated nucleic acid sequence or on the basis in SEQ ID NO: 2 indicated amino acid sequence on basis of the genetic code method or on another the person skilled in the art known manner synthesized become. Furthermore, subject matter of the instant invention are vectors, in particular cloning and/or expression vectors, comprising one of the nucleic acids according to invention specified before. The vector can be for example a prokaryotic or eukaryotic vector. The nucleic acids according to invention can become in an embodiment with flanking nucleic acids into a vector incorporated in such a way that with expression of the vector according to invention the polypeptides encoded of the nucleic acids as fusion proteins to be present or one day, a marking amino acid sequence, inertial, which can facilitate for example the cleaning and/or detection of the polypeptides. The expression vectors here preferably contain the preferred regulatory sequences suitable for the host cell, lac or the TAC promoter for an expression.

### EXAMPLES

Microorganism Identification and Verification

The microorganism used in this study was originated from Antarctic marine water near the Casey Station and named PI12 which was abbreviated from the word “psychrophilic isolate colony 12”. Upon the establishment of good growth, the stock culture was kept in 20% (v/v) glycerol and stored at −80° C.

Morphological Characteristics

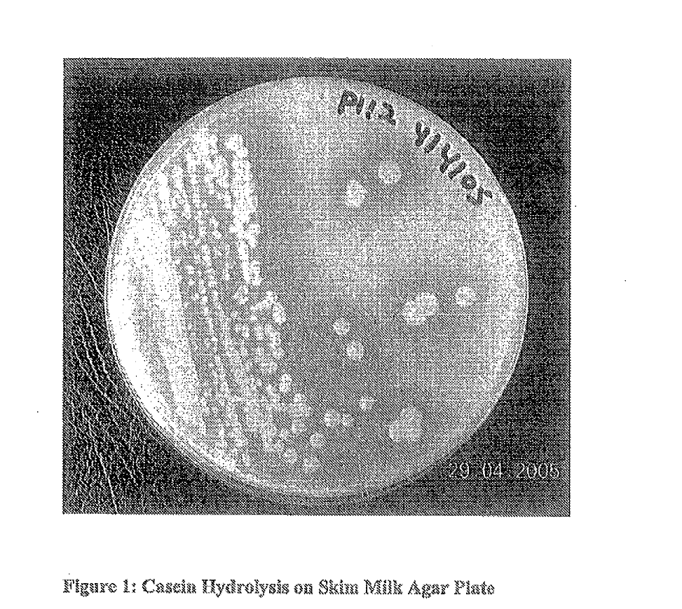

Antarctic microorganism isolated strain was grown on different types of solid media (nutrient agar, sabouroud dextrose agar and potato dextrose agar) and several antibiotics for 10 days at 4° C. and the culture characteristics (colony colour, shape, texture) were determined. Simple staining was performed to identify the cell morphology, arrangement and size of the psychrophilic isolate PI12. Scanning electron microscopy (SEM) and transmission electron microscopy (TEM) were also conducted to study the surface features and the internal ultrastructure in thin sections of the PI12 cells. The Antarctic microorganism also demonstrated resistance toward several antibiotics such as ampicillin, streptamycin and chloramphenicol. The growth for this microorganism was best at 4° C. and decreased through the temperature range tested (4° C.-20° C.) but no growth at over 20° C. FIG. 1 shows casein hydrolysis on Skim Milk Agar Plate. The Antarctic microorganism strain PI12 was grown and screened qualitatively for the protease production on skim milk agar. The plate was incubated at 4° C. for 10 days. Protease was excreted out of the cells into the surrounding media, catalyzing the breakdown of casein and formed clearing zones around the growth area.

Simple Staining

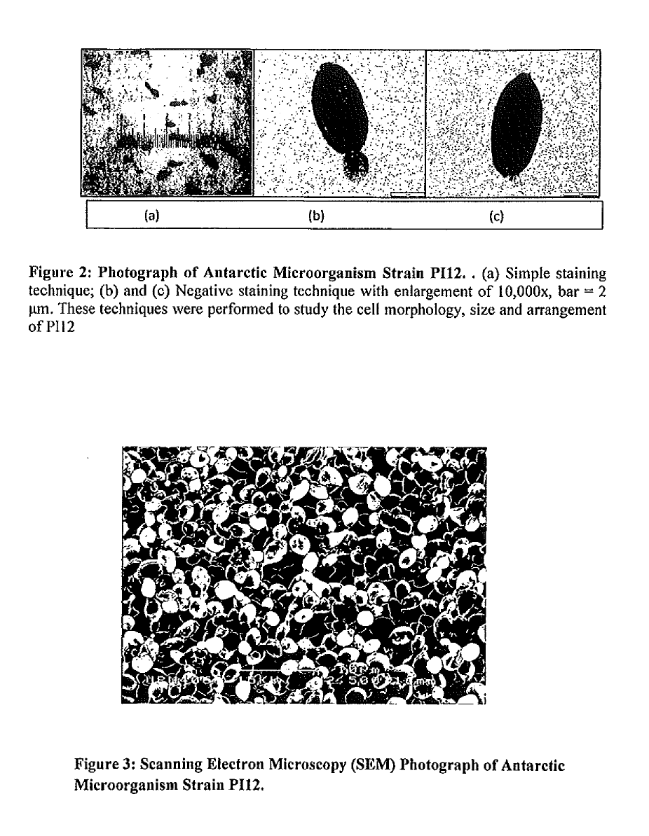

In this staining procedure, the microorganism was first fixed to a slide by the heat fixed smear. A dry clean glass slide was prepared. After flame sterilizing a loop, a loopful of culture was placed directly onto the slide and the smear was air dried. The dry slide was passed slowly through a flame three times. The smear was covered with crystal violet for at least 10 sec. Later, the slide was rinsed with water and air dry. At first, the slide was observed with low power (10×) to locate a good field. A drop of oil was added and the 100× oil immersion lens was swung into the oil by rotating the nosepiece. FIG. 2 represents the Antarctic Microorganism Strain PI12. FIG. 2 (a) represent a simple staining technique; FIGS. 2 (b) and (c) represents negative staining result of PI12 with the measured size of about 3 μm. The cells were smooth and oval. Both staining observed a budding formation of the cells indicating a yeast strain.

Scanning Electron Microscopy (SEM)

For scanning electron microscopy (SEM), the cell cultures were pelleted down at 2000 rpm for 10 min. The supernatant was discarded while the pellet was immersed in primary fixative, 4% glutaraldehyde, for 12-24 h at 4° C. (Samples may normally be stored in fixative if processing cannot be completed immediately). The sample was washed with 0.1 M sodium cacodylate buffer and placed on rocker. The procedure was repeated three times for 15 min each wash. The buffer was replaced with secondary fixative, 1% osmium tetroxide for 2 h on rocker at room temperature in a fume hood. Then, the sample was washed again with 0.1 M sodium cacodylate buffer for 3 changes of 10 min each. The sample was dehydrated in graded acetone series: 10 min each in 35%, 50%, 75% and 95%.

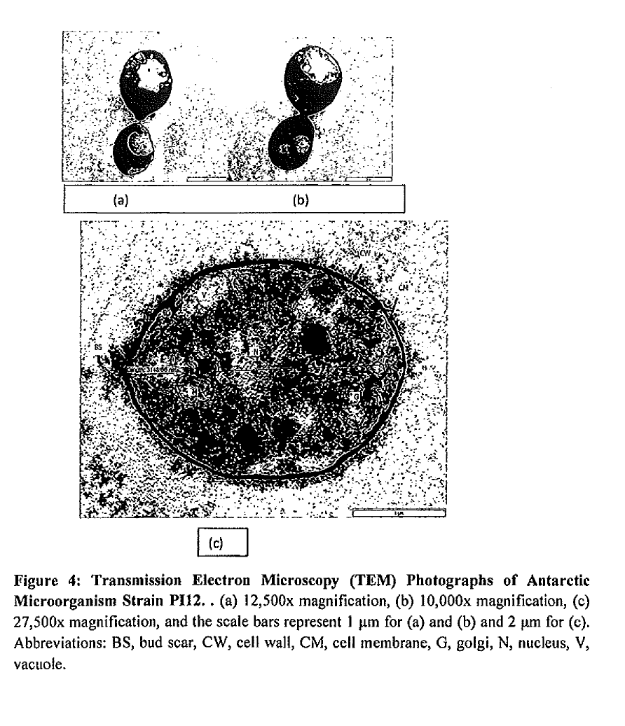

Next, dehydration in 100% acetone was performed for 3 changes of 15 min each (100% acetone for the last step should be freshly opened or stored with molecular sieves). Critical point drying process was carried out which involved the replacement of liquid in the cells with gas to create a completely dry specimen. The specimen was put into a critical dryer (Baltec CPD, Switzerland) for about 30 min. Later the specimen was mounted onto SEM stub using double sided tape or colloidal silver. The mounted specimen was coated with gold using a gold sputter coater machine (Baltec SC030, Switzerland) with ˜17-19 mA current for 3 min before they went into the SEM (Jeol JSM 6400, Japan) viewing. FIG. 3 represents an Antarctic Microorganism Strain PI12 under the magnification for the 3-D figure of budding shape microorganism is 2,500×. Bar=10 μm

Transmission Electron Microscopy (TEM)

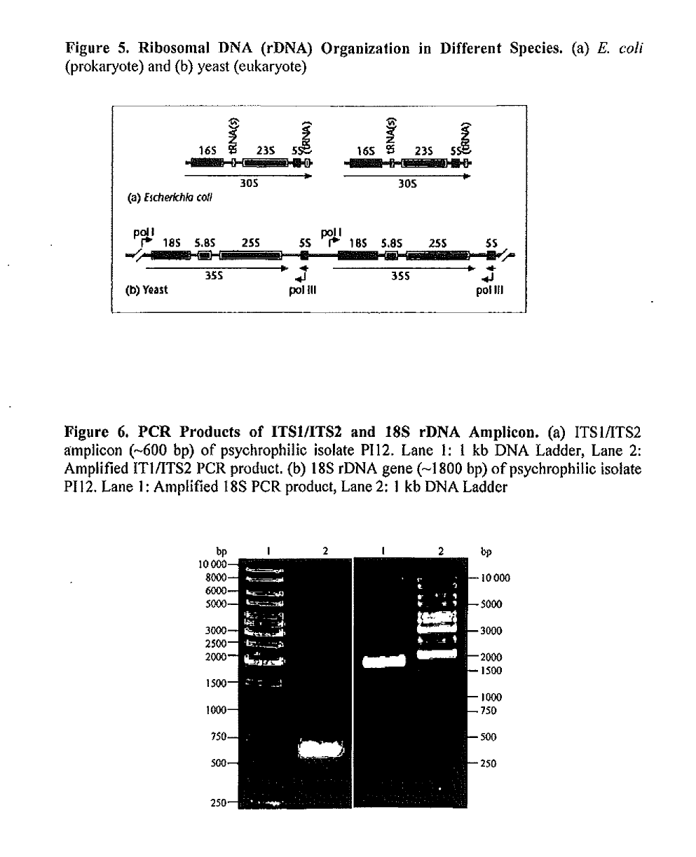

Preparation of sample for transmission electron microscopy (TEM) was the same with scanning electron microscopy (SEM) from fixation until dehydration (Refer to first paragraph of SEM) except the specimen should be cut into very small pieces, preferably 1 mm³ slices. Later, infiltration was conducted to substitute water molecule with resin. Infiltration began with 1 part resin:1 part acetone for 1 h and continued with 3 parts resin:1 part acetone for 2 h. Next, the sample was infiltrated with pure resin overnight and lastly, another 2 h with pure resin. The specimen was placed into embedding beam capsule and filled up with resin. Polymerization was held in oven at 60° C. for 1-2 days, Thick sectioning was performed using ultramicrotome to cut 1 μm thick section and placed onto glass slide. The grid was stained with toluidine blue, followed by hot plate drying and distilled water washes before viewing in TEM (Hitachi H7100, Japan). FIG. 4 represents thin-sectioned Antarctic yeast strain PI12, which reveals the intracellular organelles. FIGS. 4(a) and (b) show budding yeast with obvious appearance of membrane-bounded nucleus and easily recognized bud scar while FIG. 4(c) illustrates ultrastructure comprising of a dark and thick cell wall, a cell membrane, an irregular shape nucleus, mitochondria, vacuoles, golgi and bud scars.

Ribosomal RNA Identification

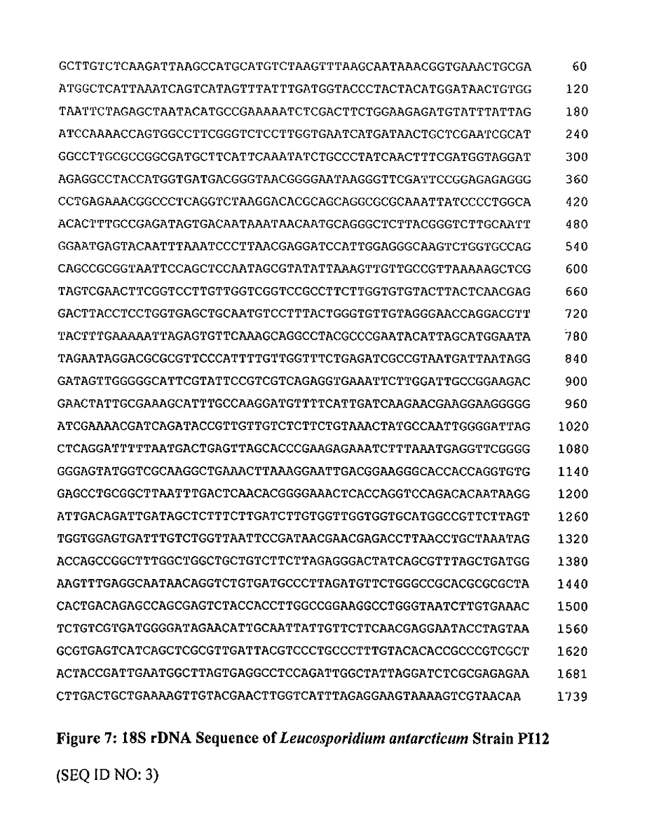

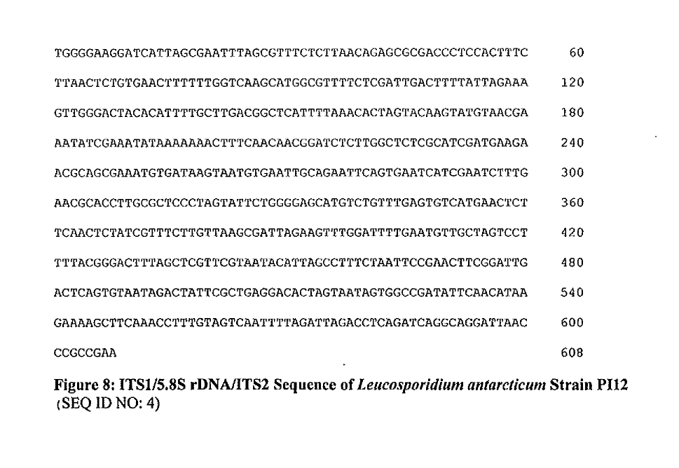

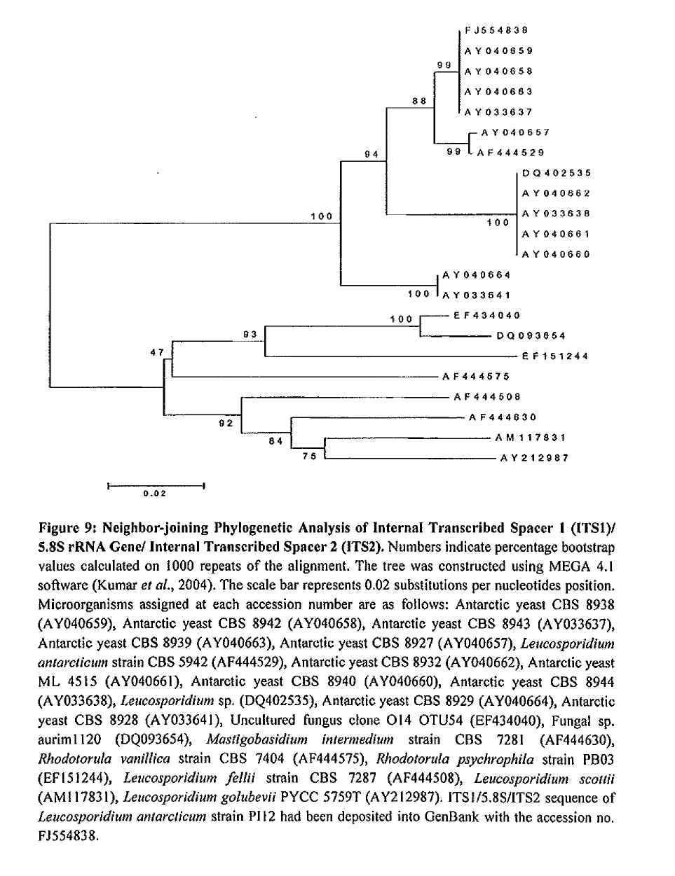

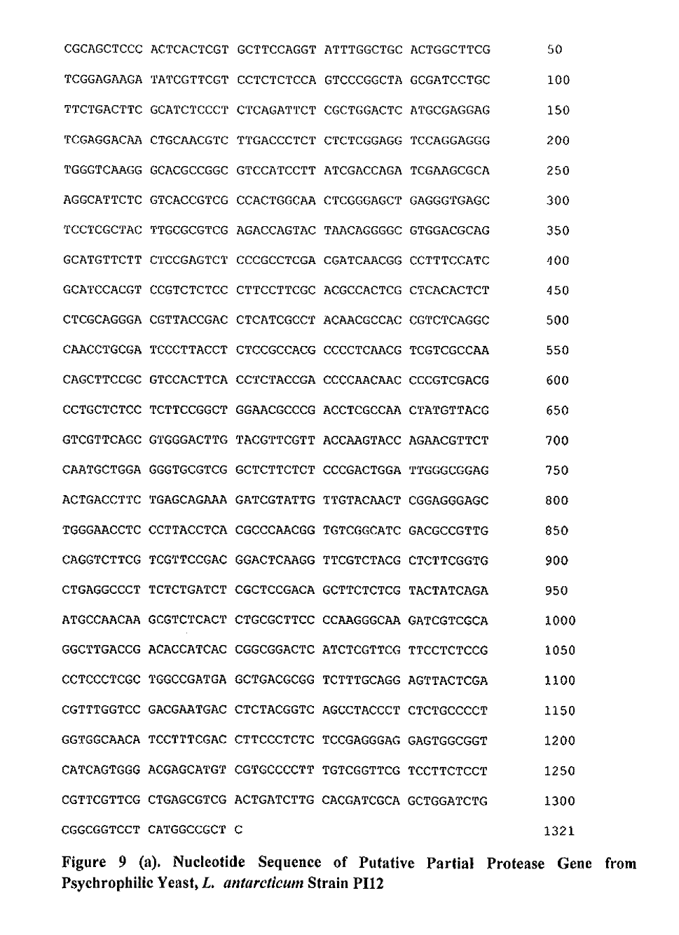

Classification and identification of unknown species was conducted through molecular approach using ribosomal RNA primers. Ribosomal RNAs are excellent molecules for discerning evolutionary relationships among microorganisms because of the ancient molecules, functionally constant, universally distributed and well conserved in sequence across broad phylogenetic distances (Suwanto, retrieved 5 Aug. 2008). 16S rRNA, 18S rRNA and ITS1/ITS2 primers were used for amplification of the putative gene via polymerase chain reaction (PCR). PCR for 16S rRNA was carried out in 50 μL of mixture containing 2.5 mM MgCl₂, 1×PCR buffer, 0.2 mM dNTP, 2 U of Taq DNA polymerase, 10 pmole of each forward and reverse primer and 50-100 ng genomic DNA. Pre-denaturation was performed at 94° C. for 4 min followed by 30 PCR cycles (94° C. 1 min, 58° C. 1 min, 72° C. 1 min). Final extension was done at 72° C., 7 min and hold at 4° C. PCR for 18S rRNA was carried out in 50 μl, of mixture containing 3.13 mM MgCl₂, 1×PCR buffer, 200 μM dNTP, 2 U of Taq DNA polymerase, 25 pmole of each forward and reverse primer and ˜150 ng genomic DNA. After 5 min pre-denaturation at 95° C., 30 PCR cycles (95° C. 1 min, 63° C. 1 min, 72° C. 2.5 min) were performed. This was followed by 1 cycle of 20 min at 72° C. and hold at 4° C. For ITS1/ITS2 region amplification, PCR was carried out in 50 μL of mixture containing 2.5 mM MgCl₂, 1×PCR buffer, 0.2 mM dNTP, 2 U of Taq DNA polymerase, 10 pmole of each forward and reverse primer and 50-100 ng genomic DNA. Pre-denaturation was performed at 94° C. for 3 min followed by 30 PCR cycles (94° C. 1 min, 58° C. 1 min, 72° C. 1 min). Final extension was done at 72° C., 7 min and hold at 4° C. The reaction was amplified in a thermocycler (GeneAmp PCR System 2400, Perkin Elmer, Foster, Calif.). The amplicons were examined by electrophoresis and sent for sequencing after being purified. A DNA homology search was performed with GenBank database (http://www.ncbi.nih.gov). FIG. 5 represents Ribosomal DNA (rDNA) Organization in Different Species. (a) E. coli (prokaryote) and (b) yeast (eukaryote). The polymerase chain reaction (PCR) technique was used to amplify the rRNA gene using several sets of primers as listed in Table 1. The 16S rRNA primers that conserved among prokaryotes were failed to amplify the desired gene. In contrast, 185 rRNA primers which conserved among eukaryotes had successfully amplified the whole region of 1739 bp of the rRNA gene [FIG. 6(b)]. Due to these findings, the Antarctic psychrophilic microorganism PI12 was classified as eukaryote. According to the 18S rDNA BLAST result, a set of primer was designed based on ITS region of Leucosporidium antarcticum strain CBS 5942 (AF444529). Soon, ITS1/5.8S rDNA/ITS2 region of Leucosporidium antarctic=strain PI12 was successfully amplified with the predicted size of 608 bp [FIG. 6(a)]. The amplicons were examined through electrophoresis and gel purified using Gel Extraction Kit (Qiagen, Germany), followed by sequencing reaction. The 18S rDNA and ITS1/5.8S rDNA/ITS2 sequence of psychrophilic yeast Leucosporidium antarcticum strain PI12 have been deposited into GenBank data library with accession number EU621372 and FJ554838, respectively. A homology search was performed with NCBI GenBank database. The 18S rDNA and ITS1/5.8S/ITS2 sequence of this psycrophilic yeast strain PI12 were listed in FIGS. 7 and 8 respectively.

TABLE 1
List of oligonucleotide pairs used for
microorganism identification

| Primer | | Sequence |
|---|---|---|
| 16S rRNA | Forward | 5′-CCGAATTCGTCGACAACAGAGTTTGATCCT-3′ |
| | Reverse | 5′-CCCGGGATCCAAGCTTACGGCTACCTTGTT-3′ |
| 185 rRNA | Forward | 5′-AACCTGGTTGATCCTGCCAGT-3′ |
| | Reverse | 5′-TGATCCTTCTGCAGGTTCACCTAC-3′ |
| ITS 1/ITS 2 | Forward | 5′-TCCGTAGGTGAACCTGCGGAAGGATCATTA-3′ |
| | Reverse | 5′-CTTTTCCTCCGCTTCTTGATATGCTTAAGT-3′ |

Characterization of Leucosporidium antarcticum Strain PI12

The Antarctic basidiomycete psychrophilic yeast strain PI12 was also identified based on physiological attributes by using the Biolog® microbial identification system (Biolog Inc., Hayward, Calif.). The Biolog YT Microplate® contained 96 wells that provide 94 biochemical tests to identify the yeast by its metabolic pattern. Each well tested the organism's ability to assimilate or oxidize a carbon source. The redox dye tetrazolium violet was used in some wells to calorimetrically indicate carbon source oxidation.

Assimilation in other wells was indicated by an increase in turbidity (Biolog, 1999). Initially wells are colourless, but if the compound in the well is assimilated and there is an increase in respiration, the cells reduce the tetrazolium dye, producing a purple color, or increased cell growth increases the turbidity of the suspension in the well (Biolog, 1999). The yeast isolate was grown on Yeast Peptone Dextrose (YPD) agar for 7 days at 4° C., streaked with a sterile swab and suspended in 12 mL of sterile water. The turbidity of the solution was adjusted to 47% transmittance by using a spectrophotometer. The yeast was placed in suspension, and the Biolog YT Microplate was inoculated with 100 mL of the suspension and incubated at 4° C.; they were checked on Biolog's Microlog® software at 24, 48 and 72 h. These intervals let a particular metabolic pattern form then interpreted by the software.

Phylogenetic Tree Analysis

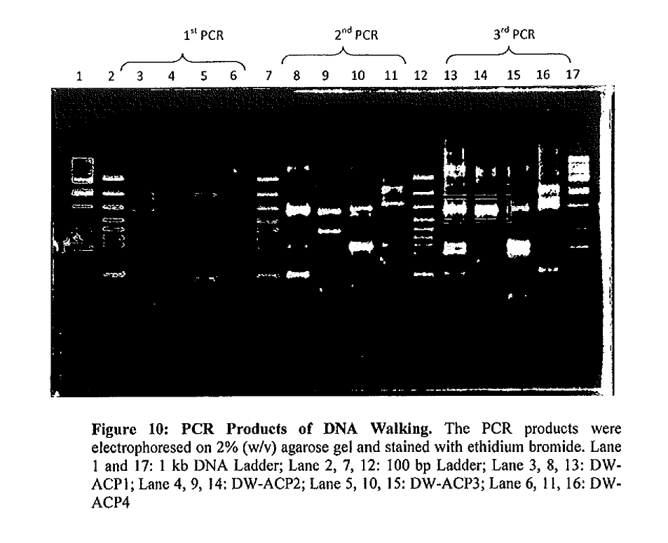

The phylogenetic tree was constructed based on comparison of ITS 1/ITS2 sequence of psychrophilic isolate 12 (PI12) yeast with the closest eukaryotic microorganisms that were extracted from GeneBank database (http://www.ncbi.nlm.nih.gov). All sequences were aligned with CLUSTALW (Multiple Sequence Alignment) that was obtained from; http://seqtool.sdsc.edu/CGI/BW.cgi and phylogenetic tree was constructed using Molecular Evolutionary Genetics Analysis (MEGA) integrated software that can be downloaded from the website (http://www.megasoftware.net). FIG. 9 represents Neighbor-joining Phylogenetic Analysis of Internal Transcribed Spacer 1 (ITS1)/5.8S rRNA Gene/Internal Transcribed Spacer 2 (ITS2). Numbers indicate percentage bootstrap values calculated on 1000 repeats of the alignment. The tree was constructed using MEGA 4.1 software (Kumar et al., 2004).

Qualitative Determination of Protease Activity

The microorganism was grown in tripticase soy broth (TSB) and streaked on skim milk agar (5%) at 4° C. for 10 days to check for protease production.

Quantitative Determination of Protease Activity

After 10 days of incubation, the supernatant was assayed for protease activity. Protease activity was determined by the modified method of Brock et al. (1982). Azocasein (0.5%, 1 mL) was dissolved in 0.1 M Tris-HCl-2 mM CaCl₂ pH 7 was pipetted into vials and pre-incubated at 4° C. The reaction was initiated by addition of 100 μL of enzyme solution for 30 min (unless stated otherwise). An equal volume of 10% (w/v) trichloroacetic acid (TCA) was added to terminate the reaction and the mixture was allowed to stand at room temperature for 30 min before centrifugation in eppendorf microcentrifuge at 13,000×g for 10 min. The supernatant was removed and mixed with an equal volume of 1 N NaOH. The absorbance was read at 450 nm using UV/Visible spectrophotometer Ultraspec 2100 pro (Amersham Biosciences, USA). One unit of azocaseinase activity was defined as the amount of enzyme activity which produces an absorbance change of 0.001 per min at 4° C. under the standard assay condition. For control, the enzyme was added at the end of the incubation period.

### Preparation of Inoculum

The bacterial inoculum was prepared by inoculating a single colony from the culture of nutrient agar into 50 mL trypticase soy broth in blue cap bottle (250 mL) and incubated at 4° C. for 10 days. The culture was harvested by centrifugation at 10,000 rpm for 10 min.

The pellet was then resuspended in 0.85% (w/v) saline to give an absorbance reading of 0.5 at 540 nm. Inoculum (5%) was then inoculated into trypticase soy broth.

### Preparation of Stock Culture

The bacterial culture was pelleted (10,000 rpm, 10 min) and resuspended in sterilized fresh medium containing 20% (v/v) glycerol for preservation at −80° C. The cultures were also inoculated into beads and nutrient agar slants which later were kept at −80° C. and 4° C. respectively to be used as working cultures throughout this project.

Obtaining Protease Gene Sequence from the Microorganism and Verifying the Gene Sequence

a) Genomic DNA Extraction

Conventional Method

A single colony of the yeast was inoculated into trypticase soy broth (TSB) (10 mL) and incubated for 10 days at 4° C. Pellet was washed two times with 1 mL GTE buffer (10 mM Tris-HCl, 1 mM EDTA, pH 8.0, 0.2% Glucose) after the cell culture (10 mL) was centrifuged at 10,000 rpm for 10 min. GTE (300 μL) was added to resuspend the pellet and the mixture was kept on ice for 5 min. RNAse (50 μL) and lysozyme (100 μL, 10 mg/mL) were added and incubated at 37° C. for 2 h. Proteinase K (50 μL, 1 mg/mL) and SDS [50 μL, 10% (w/v)] were added and proceeded with incubation at 50° C. for 30 min. Then, phenol:chloroform:isoamyl alcohol (P:C:I, 25:24:1, 500 μL) was added and mixed well by inverting the tube several times. The mixture was centrifuged at 14,000 rpm for 10 min resulting in the formation of two layers mixture. The upper layer was pipetted out, transferred into a new clean tube and the PCI-extraction step was repeated. The upper layer (200 μL) was carefully pipetted out and transferred into a new appendorf tube after the centrifugation step. Then, equal volumes of sodium acetate (NaOAc) (3 M, pH 5.5, 200 μL) and two times of isopropanol (400 μL) were added. The mixture was left at room temperature for 10 min followed by centrifugation at 14,000 rpm for 10 min. The pellet was separated out from the supernatant and washed with ethanol (500 μL). The ethanol was discarded after centrifugation at 14,000 rpm for 5 min. The pellet was dried at room temperature for 15 min, eluted in dH₂O (30 μL) and kept at −20° C. prior to experimentation.

Non-Conventional method (DNeasy Tissue Kit)

Genomic extraction was performed using the DNeasy Tissue Kit (Qiagen, Germany) according to the manufacturer's instructions.

Total RNA Extraction

RNA extraction was performed using TRIzol Reagent (Invitrogen, USA). A single colony of the yeast was inoculated into trypticase soy broth (TSB) (50 mL) and incubated for 10 days at 4° C. Mechanical disruption was carried out using liquid nitrogen. The cells were pellet down by centrifugation followed by cell lysis using TRIzol Reagent (5 mL). Chloroform (1 mL) was added followed by vigorous shaking for 15 sec. The samples was centrifuged (12,000×g) for 15 min at 2 to 8° C. Following centrifugation, the mixture was separated into a lower red, phenol-chloroform phase, an interphase and a colourless upper aqueous phase. RNA remained exclusively in the aqueous phase. The aqueous phase was transferred into a new tube, followed by precipitation of the RNA with 2.5 mL isopropyl alcohol. The sample was incubated at 15 to 30° C. for 10 min and centrifuged at 12,000×g for 10 min at 2 to 8° C. The RNA precipitate formed a gel-like pellet on the side and bottom of the tube. The RNA pellet was washed with 75% ethanol (5 mL). The sample was mixed by vortexing followed with centrifugation at 7500×g for 5 min at 2 to 8° C. The RNA was dried briefly, eluted in RNase-free water (50 μL) and kept at −80° C. prior to experimentation.

Removal of Contaminating Genomic DNA from RNA

Approximately 4 μg of DNA contaminated RNA was digested with 0.5 μg of RNAse-free DNase in a 10 μL reaction mixture containing 1× reaction buffer (20 mM Tris-HCl, pH 8.4; 50 mM KCl and 2 mM MgCl₂). The mixture was incubated at 37° C. for 15 min. DNaseI was later heat-activated at 65° C. for 15 min.

Quantitation and Quality Assessment of Genomic DNA and Total RNA

The concentration of the extracted genomic DNA and RNA was estimated spectrophotometrically using UV/Visible spectrophotometer Ultraspec 2100 pro (Amersham Biosciences, USA). Spectrophotometric quantitation was used to obtain an accurate measurement of DNA concentration. In this method, an aliquot of the genomic DNA (100 μL) was diluted in 900 μL nuclease-free dH₂O, while total RNA (100 μL) was diluted in 900 μL DEPC-treated water to give a final volume of 1000 μL, respectively. Absorbance of both DNA and RNA at 260 nm and 280 nm was measured. The ratio of A₂₆₀ to A₂₈₀ was calculated. The value of pure DNA and RNA sample should be in the range of 1.8 and 2.0. A lower ratio is an indication of protein contamination. DNA solution with A₂₆₀ contains approximately 50 μg/mL of DNA while RNA solution with A₂₆₀ contains approximately 40 μg/mL of single-stranded RNA. Thus, the concentration of DNA and RNA was calculated according to the formula below:

Concentration of DNA(μg/mL)=A₂₆₀×50 μg/mL×dilution factor

The extracted genomic DNA was analyzed with gel electrophoresis on 1% agarose gel and stained with ethidium bromide before visualized under ultraviolet radiation. A band of about 23 kb was observed. Pure DNA samples will have ratio A₂₆₀/A₂₈₀ lies between 1.8 and 2.0. The ratio of A₂₆₀/A₂₈₀ obtained was 1.8.

Analysis of PCR Products of Putative Partial PI12 Protease

Previously, seven recombinant isolates carrying putative partial protease gene were obtained using TOPO Shotgun Subcloning Kit (Invitrogen, USA). Herein, pCR 4blunt were employed as the vector and Top10 as the host. All isolates were studied for further findings. Recombinant plasmid 4blunt/PI12 protease harbouring partial putative subtilisin-like protease was chosen to proceed for further analysis. Digestion performed revealed ˜1300 bp of insert size. Purified recombinant plasmid was sent for sequencing to elucidate the sequence.

DNA Walking

DNA walking experiment of partial putative protease gene was conducted using Seegene's DNA Walking SpeedUp Premix Kit. This kit was composed of PCR Master Mix and DNA Walking Annealing Control Primers (DW-ACP) that were designed to capture unknown target sites with high specificity for up to 3000 bp long. Three target specific primers (TSP) were designed from the upstream regions of known sequence before performing the PCR reaction, with the following conditions, 18-23 nucleotides long, 40%<GC content<60%, TSP1; 55° C.≦Tm≦60° C., TSP2; 60° C.≦Tm≦65° C., TSP3. Table 2 listed the oligonucleotide primers used in the DNA walking experiment. Generally, all primers and reagents were prepared on ice before mixing and the final reaction tubes were placed in the preheated thermal cycler. The protocol of DNA walking was explained in Table 3.

TABLE 2
Oligonucleotide primers used in DNA walking experiment

| No | Oligonucleotide sequence |
|---|---|
| 1 | 2.5 μM DW-ACP1 primer for the first PCR reaction |
| | DW-ACP 1: 5′-ACP-AGGTC-3′ |
| 2 | 2.5 μM DW-ACP2 primer for the first PCR reaction |
| | DW-ACP2: 5′-ACP-TGGTC-3′ |
| 3 | 2.5 μM DW-ACP3 primer for the first PCR reaction |
| | DW-ACP3: 5′-ACP-GGGTC-3′ |
| 4 | 2.5 μM DW-ACP4 primer for the first PCR reaction |
| | DW-ACP4: 5′-ACP-CGGTC-3′ |
| 5 | 10 μM DW-ACPN primer for the second PCR reaction |
| | DW-ACP5: 5′-ACPN-GGTC-3′ |
| 6 | 10 μM Universal primer for the third PCR reaction |
| | Uni-primer: 5′-ACP-TCACAGAAGTATGCCAAGCGA-3′ |
| 7 | 10 μM TSP1 for the first PCR reaction |
| | TSP1: 5′-AGGGTCAAGACGTTGCAGT-3′ |
| | (Seq no = 178, Length = 19 mer, Tm = 55° C., GC% = 52.6) |
| 8 | 10 μM TSP2 for the second PCR reaction |
| | TSP2: 5′-ATGCGAAGTCAGAAGCAGGATC-3′ |
| | (Seq no = 114, Length = 22 mer, Tm = 60° C., GC% = 50.0) |
| 9 | 10 μM TSP3 for the third PCR reaction |
| | TSP3: 5′-GCAGCCAAATACCTGGAAGCAC-3′ |
| | (Seq no = 40, Length = 22 mer, Tm = 65° C., GC% = 54.5) |

TABLE 3
Protocol for DNA walking speedup premix kit

| | | | |
|---|---|---|---|
| PCR cocktail | Vol. | PCR setting | Cycle |

(a) First PCR

| | | | | |
|---|---|---|---|---|
| Template DNA | 5 | | 94° C. 5 min | |
| 2.5 μM DW-ACP | 4 | | 42° C. 1 min | |
| (One of DW-ACP 1, 2, 3 and | | | 72° C. 2 min | |
| 4) | | | | |
| 10 μM TSP1 | 1 | | | |
| 2x SecAmp ACP Master Mix | 25 | | 94° C. 40 sec | 30 cycles |
| II | | | 55° C. 40 sec | |
| | | | 72° C. 60 sec | |
| dH₂O | 15 | | | |
| | | | 72° C. 7 min | |
| Final | 50 | μL | 4° C. ∞ | |

(b) Second PCR

| | | | | |
|---|---|---|---|---|
| Purified first PCR product | 2 | | 94° C. 5 min | |
| 10 μM DW-ACPN | 1 | | | |
| 10 μM TSP2 | 1 | | 94° C. 40 sec | 35 cycles |
| 2x SeeAmp ACP Master Mix II | 10 | | 60° C. 40 sec | |
| | | | 72° C. 60 sec | |
| dH₂O | 6 | | | |
| | | | 72° C. 7 min | |
| Final | 20 | μL | 4° C. ∞ | |

(c) Third PCR

| | | | | |
|---|---|---|---|---|
| Second PCR product | 1 | | 94° C. 5 min | |
| 2.5 μM Universal primer | 1 | | | |
| 10 μM TSP3 | 1 | | 94° C. 40 sec | 30 cycles |
| 2x SeeAmp ACP Master Mix II | 10 | | 60° C. 40 sec | |
| | | | 72° C. 60 sec | |
| dH₂O | 7 | | | |
| | | | 72° C. 7 min | |
| Final | 20 | μL | 4° C. ∞ | |

Note:
The first PCR products were purified using PCR purification Kit (Qiagen, Germany)

As a result, a total length of 2687 bp putative protease gene was successfully harboured. However, the putative gene was predicted to contain several introns since it was amplified using genomic DNA of yeast (eukaryotic gene). RT-PCR and RACE were accomplished to verify the hypothesis and to acquire the full-length of the protein.

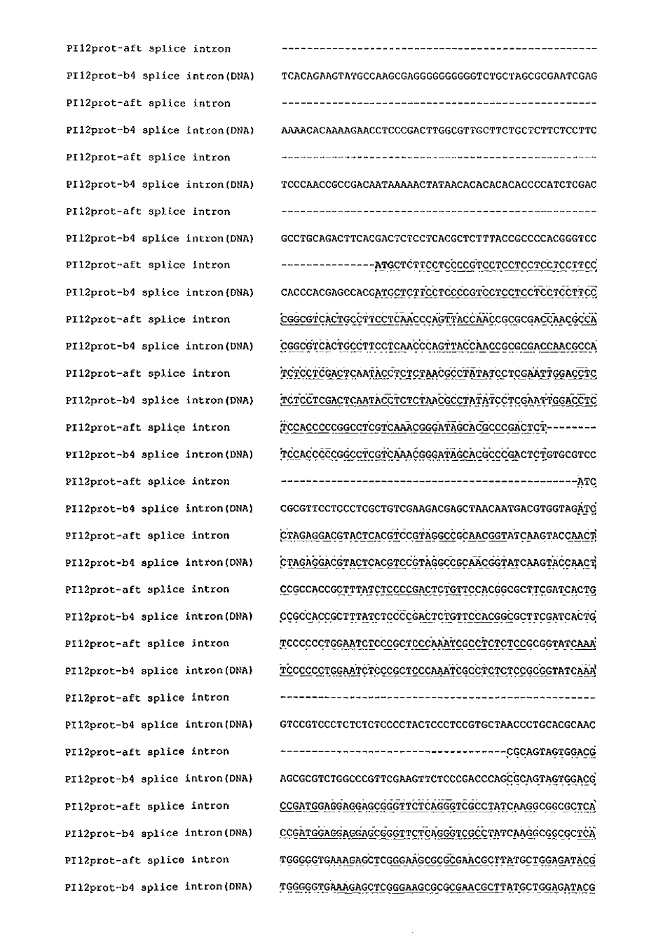

FIG. 10 shows the PCR products of DNA walking after first, second and third PCR reactions. Multiple bands were amplified after third PCR reaction where all bands could be the target bands since the expected size was unknown. In general, target bands might not be shown in first gel but always shown in second and third gel. The target bands were discriminated by comparing the size of bands between second and third PCR. A few cleaned and intensed bands with predicted product sizes were chosen and cloned into pBAD TOPO TA expression vector (Invitrogen, USA) followed by sequencing reaction.

Sequencing of DNA Walking PCR Products

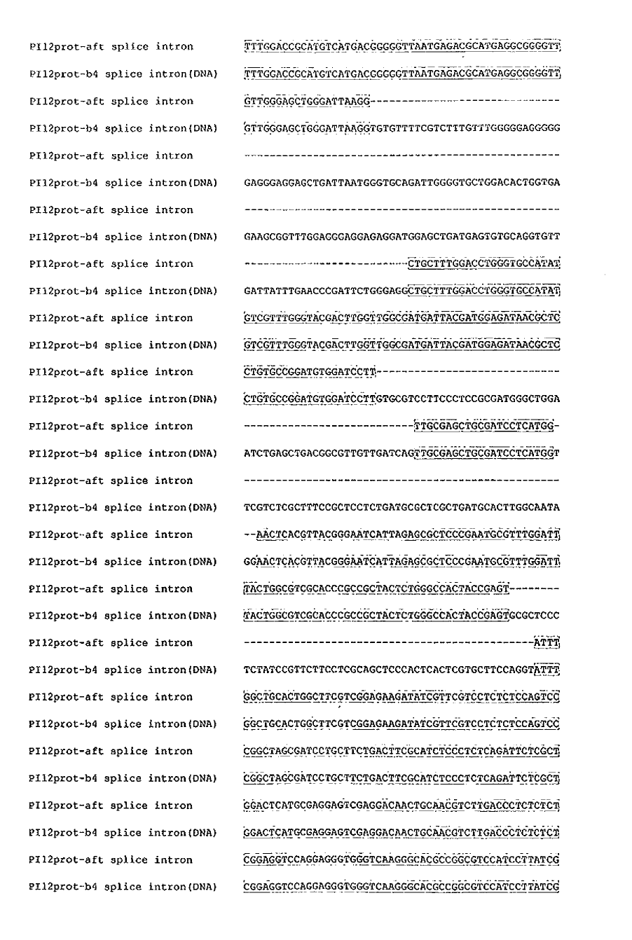

Several putative PCR products derived from DNA Walking amplification were cloned into pBAD TOPO vector. The recombinant plasmid 4blunt which carrying putative partial PI12 protease gene (˜1300 bp) and recombinant plasmid pBAD/DW-ACP1 (3), bearing the putative upstream region of the partial PI12 protease gene (˜1500 bp), amplified through DNA walking were sent for sequencing using the designed primers. The DNA sequences obtained were analyzed using Basic Local Alignment Search Tool (BLAST) program from National Center of Biotechnology Information (NCBI) (http://www.ncbi.nih.gov) and translation was performed with Expasy Molecular Biology Server (http://www.expasy.heugec.ch) while homology similarity was checked through the database in Biology Workbench (http://biology.ncsa.uiuc.edu). FIG. 11 represents Alignment between Putative PI12 Protease Gene Sequence from DNA Walking and Predicted ORF Sequence. The alignment which was performed by Augustus webserver showing position of putative start and stop codon, comparison and location of introns and exons in the sequence. Nucleotide sequence analysis by Augustus Web server revealed a putative Open Reading Frame (ORF) of 2892 bp in length which codes for 963 amino acids with molecular mass and pI of 100.99 kDa and 6.41, respectively. This cold-adapted serine protease has been deposited into GenBank with accession no. CAQ76821. This is the second extracellular protease reported being synthesized by this obligate psychrophilic yeast, L. antarcticum and interestingly no published report on recombinant protease from this L. antarcticum has been described to date. In addition, this is a new breakthrough since this huge serine protease has a very few identity with other proteases and no homology similarity with other psychrophilic protease Sequence analysis through bioinformatics studies revealed novel discoveries about this protein and it is particularly interesting enzyme since very few information had been unveiled from cold-adapted yeast until now.

Reverse-Transcriptase Polymerase Chain Reaction (RT-PCR)

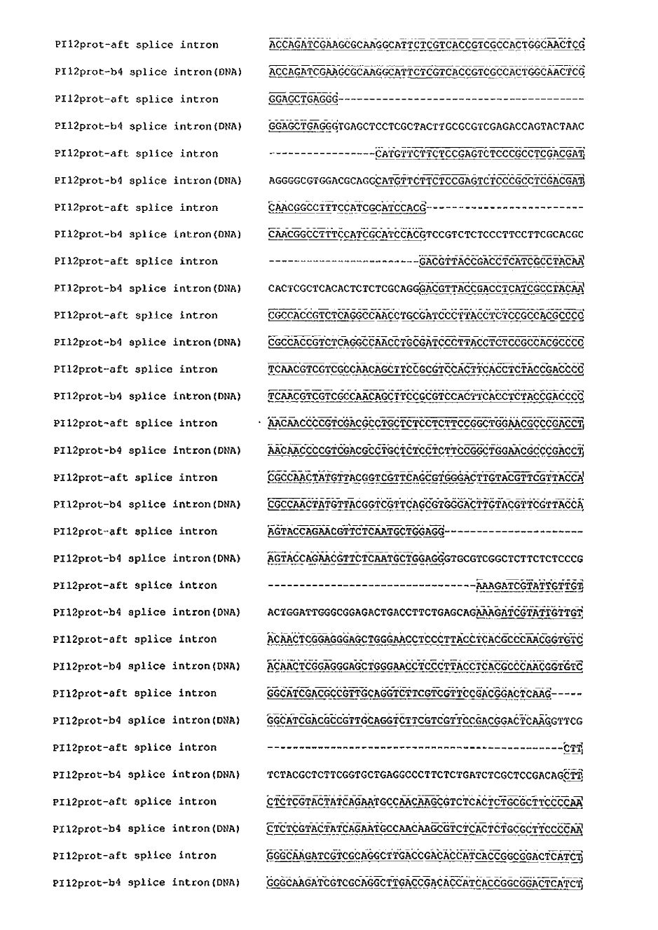

Reverse-transcriptase polymerase chain reaction (RT-PCR) was performed using the SuperScript™ III First-Strand Synthesis System (Invitrogen, USA). cDNA was is synthesized in the first step using total RNA of Leucosporidium antarcticum strain PI12 primed with oligo (dT). In the second step, PCR was performed in a separate tube using target specific primers to amplify the gene of interest. First-strand cDNA synthesis was carried out in 10 μL of mixture containing 5 μg total RNA, 50 μM Oligo (dT)₂₀, 10 mM dNTP mix and DEPC-treated water. The mixture was incubated at 65° C. for 5 min. The mixture was placed on ice for about 1 min while preparing the cDNA synthesis mix consisted of 10× reverse transcriptase (RT) buffer (200 mM Tris-HCl, (pH 8.4), 500 mM KCl), 25 mM MgCl₂, 0.1 M DTT, 40 U/μL RNaseOUT and 200 U/μL SuperScript III RT. 10 μL of the cDNA synthesis mix was added into the RNA mixture followed by incubation at 50° C. for 50 min. The reaction was terminated by incubation at 85° C. for 5 min followed by a brief centrifugation. 1 μL of RNase H was added into the mixture and incubated at 37° C. for 30 min, followed by amplification of target cDNA. PCR was carried out in 50 μl, of mixture containing ˜150 ng cDNA, 3.125 mM MgCl₂, 200 μM dNTP, 2 U of Tag DNA polymerase, 1×PCR buffer (MBI, Fermentas) and 25 pmole of each forward (5′-ATGCTCTTCCTCCCCGTCCTCCTCCT-3′) and reverse (5′-TCAGCGAACGAACGAGGAGAAGGT-3′) primer. After 4 min pre-denaturation at 95° C., 30 PCR cycles (95° C. 1 min, 58° C. 1 min, 72° C. 1 min) were performed. This was followed by 1 cycle at 72° C., 7 min and hold at 4° C. The reaction is amplified in a thermocycler (GeneAmp PCR System 2400) and later examined by electrophoresis. FIG. 12 represents PCR Products of Putative Protease Gene using Genomic DNA and cDNA Template of L. antarcticum Strain PI12. Lane 1: 1 kb DNA Ladder; Lane 2: -ve control (no RT); Lane 3-5: PCR product, cDNA as template (predicted size: 1.6 kb); Lane 6: PCR product, DNA as template (predicted size: 1.8 kb)

First-Strand cDNA Synthesis

Prior to full-length cDNA amplification of the PI12 protease, rapid amplification of cDNA ends (RACE) was carried out using the SMART™ RACE cDNA Amplification Kit (Clontech, USA). Initially, first-strand cDNA was synthesized to be the template for RACE. Two separate cDNA populations; 5′-RACE-Ready cDNA and 3′-RACE-Ready cDNA were synthesized using the SMART RACE Kit technology. 50 ng-1 μg of total RNA was needed for the production of optimum yield of first-strand cDNA. For the preparation of 5′-RACE-Ready cDNA, 1 μL of 5′-CDS primer A and 1 μL of SMART II A oligo were added to 3 μL RNA sample while for the preparation of 3′-RACE-Ready cDNA, 1 μL of 3′-RACE CDS primer A was added to 3 μL RNA sample and sterile dH₂O was added to a final volume of 5 μL for each reaction. Shortly, the contents were mixed and spin briefly in a microcentrifuge. The tubes were incubated at 70° C. for 2 min and were cooled on ice for 2 min. The following were added to each reaction tube which made up a total volume of 10 μl; 2 μL 5× first-strand buffer (250 mM Tris-HCl (pH 8.3) 375 mM KCl, 30 mM MgCl₂), 1 μL DTT (20 mM), 1 μL dNTP Mix (10 mM), 1 μL MMLV SuperScript II reverse transcriptase (200 U/μL) (Invitrogen, USA). The contents of the tubes were mixed by gently pipetting and spin briefly to collect the contents at the bottom. The tubes were incubated at 42° C. for 1.5 h in a heating block. The first-strand reaction product was diluted with 100 μL of Tricine-EDTA buffer [10 mM Tricine-KOH (pH 8.5), 1.0 mM EDTA] since the starting total RNA was >200 ng. Lastly, the tubes were heated at 72° C. for 7 min. The samples were stored at −20° C. or directly proceed with RACE reaction.

Rapid Amplification of cDNA Ends (RACE)

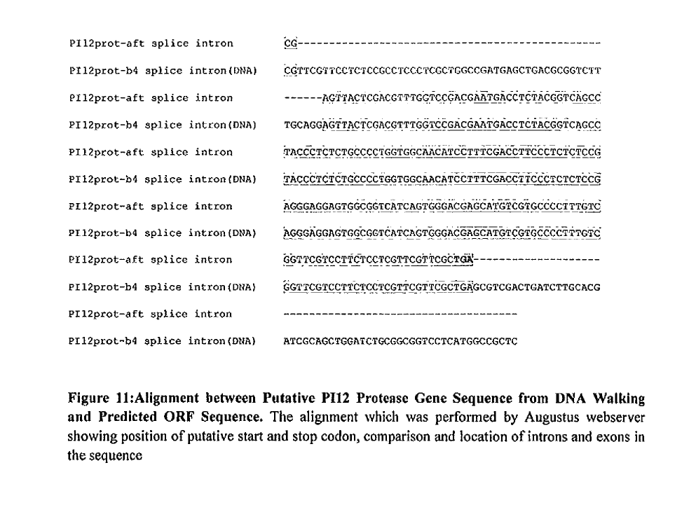

PCR master mix was prepared for both 5′-RACE and 3′-RACE PCR reactions to generate the 5′ and 3′ cDNA fragments. For each 50 μL PCR reaction, the following reagents were mixed; 34.5 μL PCR grade water, 5 μL 10× Advantage 2 PCR buffer (400 mM Tricine-KOH (pH 8.7), 150 mM KOAc, 35 mM Mg(OAc)₂, 37.5 μg/mL BSA, 0.05% Tween 20, 0.05% Nonidet-P40), 1 μL dNTP Mix (10 mM) and 1 μL 50× Advantage 2 Polymerase Mix. Mix well both tubes by vortexing (without introducing bubbles), and then the tubes were spun briefly in a microcentrifuge. PCR cocktails for both 5′-RACE and 3′-RACE were prepared by adding 2.5 μL 5′-RACE-Ready cDNA and 3′-RACE-Ready cDNA as the template together with 1 μL Gene Specific Primer 1 (GSP 1) and Gene Specific Primer 2 (GSP 2) into the PCR master mix, respectively. 5 μL of 10× Universal Primer A Mix was added to both reactions which made up a final volume of 50 μL. Details about the primers were listed in Table 4. Thermal cycling is commencing using a touchdown PCR technique (Roux, 1995; Don et al., 1991) which significantly improves the specificity of SMART RACE amplification. PCR was conducted in 3-step cycles where firstly, 5 cycles of denaturation (94° C., 30 sec) followed by extension (72° C., 3 min) was commenced. Later, another 5 cycles of denaturation (94° C., 30 sec), annealing (70° C., 30 see) and extension (72° C., 3 min) was performed. Finally, the PCR reaction is completed with another 30 cycles of reaction consisted of denaturation (94° C., 30 sec), annealing (68° C., 30 sec) and extension (72° C., 3 min). The PCR tubes were preserved at 4° C. Applying the RACE strategy had fruitfully amplified two intact and sharp 5′-RACE and 3′-RACE bands with the size of ˜750 bp and ˜1500 bp, respectively (FIG. 13). Both of the PCR products were purified, cloned into pJET1.2 blunt vector (Fermentas, USA) and sequenced to obtain the sequences of the extreme ends of the transcript.

TABLE 4
Oligonucleotide primers used in first-strand
cDNA synthesis and RACE reaction

| No | Oligonucleotide sequence |
|---|---|
| 1 | SMART II ™ A Oligonucleotide (12 μM) |
| | 5′-AAGCAGTGGTATCAACGCAGAGTACGCGGG-3′ |
| 2 | 5′-RACE CDS Primer A (5′-CDS; 12 μM) |
| | 5′-(T) 25V N-3′ |
| 3 | 3′-RACE CDS Primer A (3′-CDS; 12 μM) |
| | 5′-AAGCAGTGGTATCAACGCAGAGTAC(T) 30V N-3′ |
| 4 | 10x Universal Primer A Mix (2 μM) |
| | 5′-CTAATACGACTCACTATAGGGC-3′ |
| 5 | Gene Specific Primer 1 (GSP1) (10 μM) |
| | 5′-ACCAGTGTCCAGCACCCCAATCTTAATCC-3′ |
| | (Length = 29 mer, Tm = 68.9° C., GC% = 51.7) |
| 6 | Gene Specific Primer 2 (GSP2) (10 μM) |
| | 5′-TCATCAGTGGGACGAGCATGTCGT-3′ |
| | (Length = 24 mer, Tm = 63.7° C., GC% = 54.2) |

Note:
N = A, C, G, or T;
V = A G, or C

Full-Length cDNA Amplification

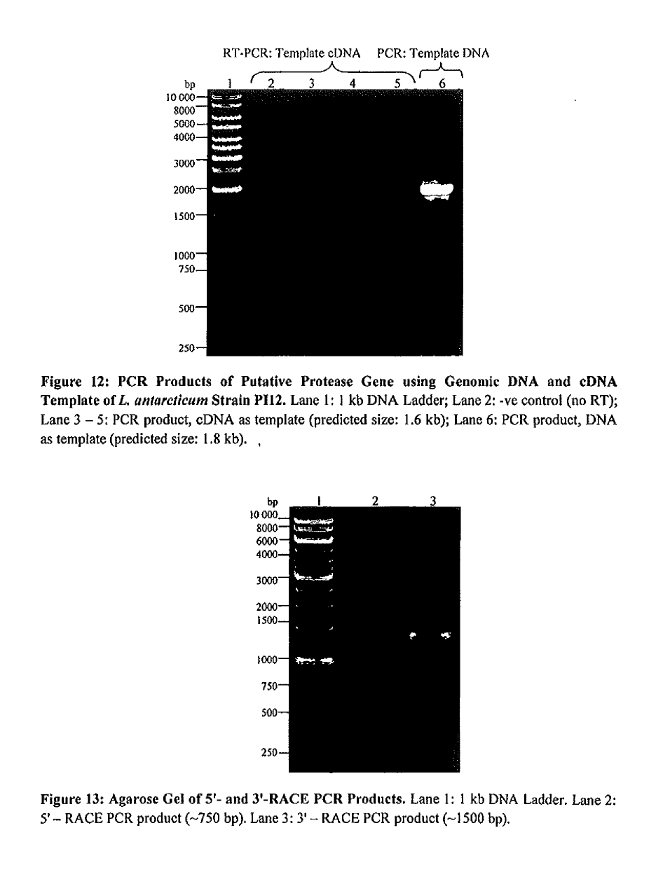

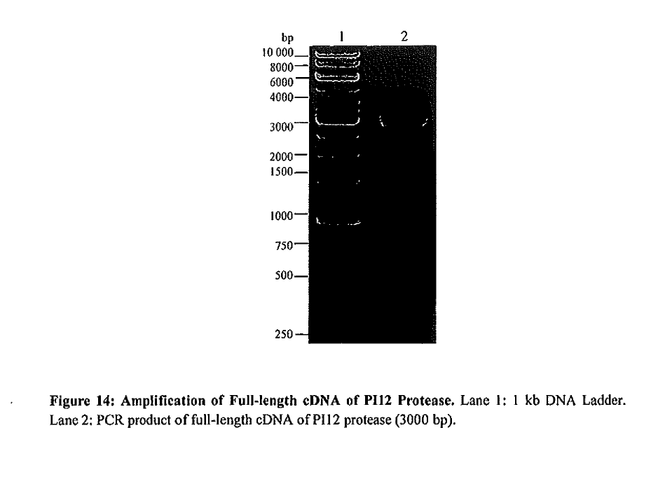

Rapid amplification of cDNA ends (RACE) produced two PCR products (5′-RACE and 3′-RACE) which were cloned and sequenced to capture the end-to-end sequence of both products. Later, 1 set of primer was designed from the extreme 5′ and 3′ ends of cDNA (For 5′ CAP: 5′-GCGGGGGCCGACAATAAAAAC-3′ and Rev 3′ A-Tail: 5′-TTTTTTTTTTTTTTTTTTTTTTTTTTGAGGTGGC-3′) whereas the 5′-RACE-Ready cDNA served as the template to generate full-length cDNA through long distance PCR (LDPCR). Amplification process was carried out in a reaction mixture (50 μL) containing 5 μL of 50-100 ng DNA template, 1 μL (10 pmole/μL) of each forward and reverse primers, 1 μL of 50×dNTP mix, 1 μL of 1 U/μL 50× Advantage 2 polymerase mix, 5 μL of 10× Advantage 2 PCR buffer (Clontech, USA) and 36 μL of distilled water. Pre-denaturation was performed at 95° C. for 1 min followed by 30 PCR cycles of denaturation (95° C., 30 see), annealing (61° C., 3 min) and extension (68° C., 3 min). Final extension was conducted at 68° C. for 3 min and hold at 4° C. The gene was amplified using thermocycler (CG 1-96 Corbett Research, Australia). The amplicon was examined by electrophoresis and cloned into pJET1.2 blunt vector (Fermentas, USA). After being purified, the recombinant plasmid was sent for sequencing and sequence analysis was conducted using several publicly available webservers. FIG. 14 represents amplification of Full-length cDNA of PI12 Protease. Lane 1: 1 kb DNA Ladder. Lane 2: PCR product of full-length cDNA of PI12 protease (3000 bp). Nucleotide and deduced amino acid sequences of genomic DNA and cDNA fragment encoding PI12 protease were shown in FIG. 15. A single Open Reading Frame (ORF) comprised 2892 bp (without introns) that coded for a protein of 963 amino acids was observed. The predicted molecular mass and pI were 100.99 kDa and 6.41 respectively. Translation starts at a nucleotide position 1 and translation stop is marked with an asterisk. The nucleotide sequences atg (1 to 3), and tag (3598 to 3600) indicate the initiation codon and terminal codon, respectively. PI12 protease is therefore belongs to the subtilisin subgroup of the subtilase serine protease superfamily. This cold-adapted serine protease has been deposited into GenBank with accession no. CAQ76821. This is the second extracellular protease reported being synthesized by this obligate psychrophilic yeast, L. antarcticum and interestingly no published report on recombinant protease from this L. antarcticum has been described to date. In addition, this is a new breakthrough since this huge serine protease has a very few identity with other proteases and no homology similarity with other psychrophilic protease Sequence analysis through bioinformatics studies revealed novel discoveries about this protein and it is particularly interesting enzyme since very few information had been unveiled from cold-adapted yeast until now.

Cloning of Protease Gene into Expression Vector

a) Preparation of Escherichia coli Competent Cell

E. coli strain Top10 was used for propagation and cloning of the empty vector and recombinant vector. The competent cells were prepared based on rubidium chloride method (Sambrook et al., 1989). A single colony of E. coli Top10 was inoculated in 10 mL of LB broth and grown at 37° C. with agitation at 200 rpm for overnight. 1 mL of the culture was transferred into 50 mL of LB medium in a 250 mL flask and incubated at 37° C. until OD₆₀₀ of 0.5 was obtained. The cells were pelleted at 4000×g for 15 min at 4° C. and the supernatant was discarded gently. A chilled TFB1 buffer containing 100 mM RbCl₂, 50 mM MnCl₂, 30 mM KAc, 10 mM CaCl₂ and 15% (v/v) glycerol was added to the cells (15 mL for a 50 mL culture). The suspension was kept on ice for 1 h. The cells were collected by centrifugation (4000×g for 15 min at 4° C.) and the pellet were resuspended in 2 mL ice-cold filter-sterilized TFB2 buffer containing 10 mM PIPES, 10 mM RbCl₂, 75 mM RbCl₂, 75 mM CaCl₂ and 15% (v/v) glycerol (adjusted to pH 6.8 with KOH). Aliquots of 100 μL were prepared in sterile microcentrifuge tubes and stored at −80° C.

b) Amplification of Mature PI12 Protease Gene

The gene encoding mature PI12 protease was amplified by PCR using recombinant pJET1.2/FLcDNA of PI12 protease as template. Forward primer (Forward AvrII: 5′-CAAGCCCTAGGCTACGCACGGAACGAGAA-3′) and reverse primer (Reverse EcoRI: 5′-CTGCCGAATTCTTCCTCAACCCAGTTACCAAC-3′) with AvrII and EcoRI restriction sites (underlined) were designed based on mature PI12 protease gene sequence (Accession no. FM178559) for flanking the PCR product at the 5′- and 3′-terminus, respectively. The restriction sites were chosen based on the multiple cloning sites (MCS) of the plasmid pPIC9 (Appendix C). Amplification process was carried out in a reaction mixture (50 μL) containing 5 μL of 50-100 ng DNA template, 1.5 μL (10 pmole/μL) of each forward and reverse primers, 1 μL of 10 mM dNTP mix, 4 μL of 25 mM MgCl₂, 1 μL of 1 U/μL Tag DNA polymerase, 5 μL of 10×PCR buffer (MBI, Fermentas, USA) and 31 μl of dH₂O. Preliminarily, PCR was commenced by pre-denaturation step at 94° C. for 4 min followed by 30 PCR cycles of denaturation (94° C., 1 min), annealing (65° C., 1 min) and extension (72° C., 2 min). Final extension was conducted at 72° C. for 7 min and hold at 4° C.

c) Cloning of Protease Gene into Pichia Expression Vectors

The PCR product of the PI12 protease gene and the vector pPIC9, (0.5-1 μg) were separately added to restriction enzyme digestion mixtures containing 3 μL EcoRI (10 U/μL), 6 μL AvrII (10 U/μL), 6 μL Y⁺ Tango buffer (10×) and dH₂O to a final volume of 30 μL. The reactions were incubated at 37° C. for 1 h. The digestion products were observed through agarose gel electrophoresis and gel purified using QIAquick Gel Extraction Kit (Qiagen, Germany). The construct was added to a ligation mixture with a ratio of 1:5 to pPIC9 vector. The ligation mixture was comprised of 1 μL of 10× ligation buffer (MBI Fermentas, USA), 1 μL T4 DNA Ligase (5 U/μL; MBI Fermentas, USA) and dH₂O to a final volume of 10 μL. The reaction was incubated overnight (14-16 h) at 16° C.

d) Heat-Shock Transformation of Escherichia coli

The procedure for heat-shock transformation was performed according to the method described by Sambrook et al. (1989). The ligation mixture (10 μL) was added to 200 μL of E. coli competent cells in a sterile 1.5 mL microcentrifuge tube and was kept on ice for 20-30 min. This tube was then heat shocked for 60 sec at 42° C. followed by chilling briefly on ice (2 min). LB broth was added to the transformation mixture and was incubated at 37° C. for 1 h with agitation at 200 rpm. Finally, 50-100 μL of the transformation mixture was spread on LB agar containing 100 μg/mL ampicillin. The plates were incubated overnight at 37° C. The transformants were selected for further studies.

e) Analysis of Recombinant Plasmids

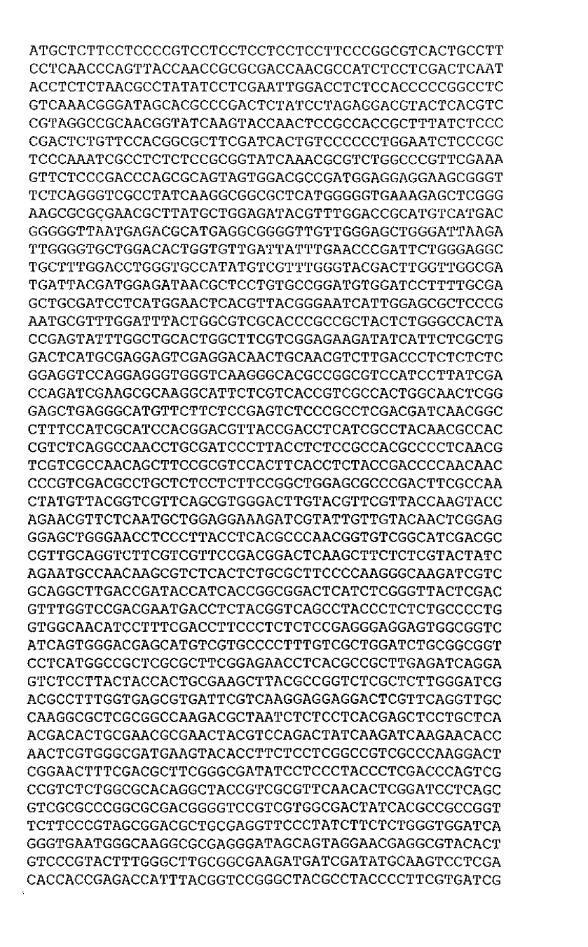

The transformants were inoculated into 10 mL of LB broth supplemented with 100 μg/mL ampicillin and grown overnight at 37° C. with agitation at speed 200 rpm. The plasmids were extracted using QIAprep Spin Miniprep Kit (Qiagen, Germany) according to the manufacturer's instructions. Digestion with restriction enzymes (EcoRI and AvrII) was carried out on the recombinant plasmids to verify the presence of the insert. Analysis of restriction enzyme digestion was done in a reaction mixture as follows: 4 μL plasmid DNA (0.5 μg) 1 μL EcoRI (10 U/μL), 2 μL AvrII (10 U/μL), 2 μL Y⁺ Tango buffer (10×) and dH₂O to a final volume of 10 μL. The digested products were analyzed by 1% (w/v) agarose gel electrophoresis as mentioned previously. Size of the digested DNA was estimated based on the 1 kb DNA marker (MBI Fermentas, USA). FIG. 16 represents analysis of Recombinant Plasmid, pPIC9. Lane 1: 1 kb DNA Ladder. Lane 2: Circular form of empty plasmid (control). Lane 3: Circular form of recombinant plasmid. Lane 4: Single-digested recombinant plasmid with EcoRI and Lane 5: Double-digested recombinant plasmid with EcoRI and AvrII.

f) Sequencing and Glycosylation Site Prediction

The recombinant plasmid pPIC9/mature PI12 protease was sent for sequencing by automated sequencer (First Base, Malaysia) to confirm that the PI12 protease gene is in frame with the N-terminal α-factor signal sequence of the plasmid. The primers used were based on the α-factor signal sequence (5′-TACTATTGCCAGCATTGCTGC-3′) and the AOX1 promoter (For 5′:5′-GACTGGTTCCAATTGACAAGC-3′ and Rev 3′:5′-GCAAATGGCATTCTGACATCC-3′) of the plasmid pPIC9. The DNA sequence of the cloned PI12 protease gene was analyzed and compared with the submitted PI12 protease sequence (FM178559) using the Biology Workbench 2.0 (http://workbench.sdsc.edu/). N-glycosylation sites of recombinant PI12 protease were predicted using the on-line predicted server NetNGlyc version 1.0 (http://www.cbs.dtu.dk/services/NetNGlyc/), which predicts N-glycosylation sites in proteins by using artificial neural networks that examined the sequence context of Asn-Xaa-Ser/Thr sequons (Gupta and Brunak, 2002).

Cloning of PI12 Protease Gene in Pichia pastoris

a) Transformation of Recombinant Plasmid into Pichia pastoris

Prior to transformation into P. pastoris cell, the empty pPIC9 and pPIC9 recombinant plasmids harbouring PI12 protease gene were linearized using PmeI (10 U/μL) restriction enzyme digestion. The digestion mixture consisted of 2.5 μL Y⁺ Tango buffer (10×), 1 μL of PmeI, and 10 μL plasmids while dH₂O was added to a final volume of 25 μL. The linearized plasmids were purified using QIAquick PCR Purification Kit.

b) Preparation of Pichia pastoris Competent Cells

The preparation of electrocompetent cells was done according to Pichia Expression Kit Manual (Invitrogen, USA). A single colony of P. pastoris strain GS 115 and KM71 were inoculated in YPD media [10 mL) (1% (w/v) yeast extract, 2% (w/v) peptone, 2% (w/v) glucose] and grown at 30° C. with agitation at 250 rpm for 16-30 h. 1 mL of the cultures were transferred in 25 mL of fresh YPD broth and were grown in the conditions mentioned above until the cell density reached at A₆₀₀ nm˜1.3-1.5. The cultures were chilled on ice for 10 min and centrifuged at 1500×g, 4° C. for 5 min. The pellets were resuspended in an equal volume of sterile and chilled dH₂O. The cells were centrifuged at 1500×g, 4° C. for 5 min and the pellets were resuspended in 0.5 volume of ice-cold sterile dH₂O. The cells were then resuspended in 10 mL of 1 M sorbitol. About 80-100 μL of the cells was transferred into an overnight pre-chilled (kept in −20° C.) 0.2 cm electroporation cuvette (BioRad, USA) and mixed with 10 μL (1-5 μg) recombinant DNA.

c) Transformation into Pichia pastoris Cells Via Electroporation

The transformation mixtures (mixture of cells and plasmids) were incubated on ice for 5 min in the electroporation cuvette and pulsed with an electroporator (Gene Pulser, BioRad, USA) at following parameters: charging voltage of 1500 V, capacitance of 25 μF and resistance of 400Ω, which generated a pulse length of ˜5-10 ms with a field strength of ˜7500 V/cm. Iced-cold 1 M sorbitol (1 mL) was added immediately after the pulse into the cuvettes. The content was transferred into a sterile bijou bottle and incubated at 30° C. for 2 h, without shaking. Later, 10-200 μL of the cells was spread on the MD agar [Minimal dextrose medium; 1.34% (w/v) yeast nitrogen base (YNB), 4×10^{-5%} (w/v) biotin, 2% (w/v) dextrose, 2% (w/v) agar]. The plates were incubated at 30° C. for 3-5 days until the transformant colonies formed.

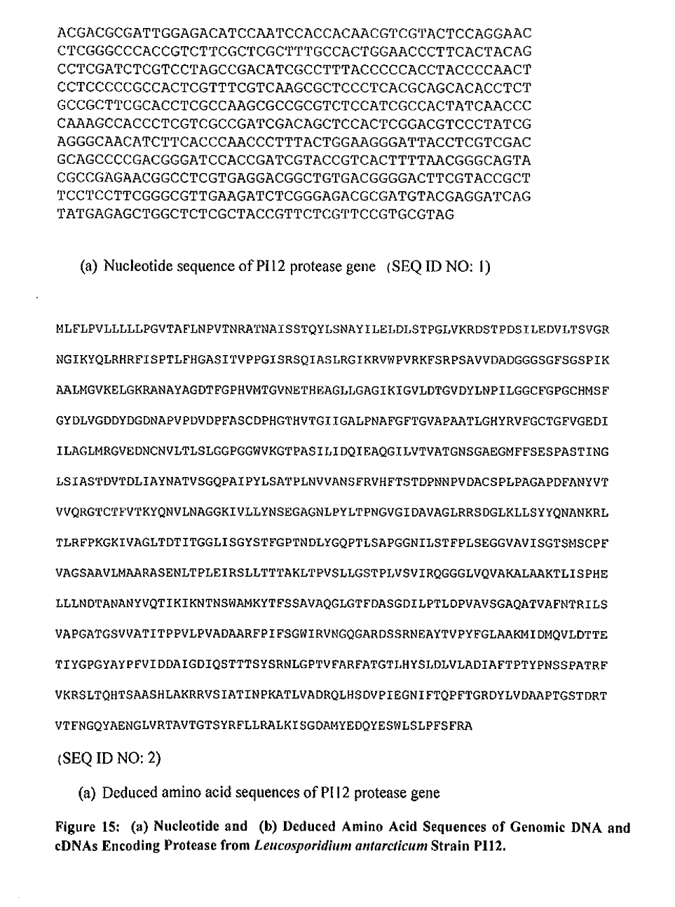

The recombinant pPIC9/PI12 protease was then linearized using PmeI to stimulate recombination when transformed into P. pastoris. Integration event between the vector and Pichia genome that held at the 5′AOX1 locus is a proficient undemanding way to generate recombinant clones for heterologous protein expression. Linearization of the plasmid resulted in single DNA band representing the total size of pPIC9 with PI12 protease (FIG. 17). Through electroporation, more than 100 colonies per plate of Pichia transformants of both strains (GS115 and KM71) were obtained on the Minimal dextrose agar (MD agar; 1.34% yeast nitrogen base, 4×10⁻⁵% biotin, 2% dextrose and 2% agar) after 5 days of incubation in 30° C. Minimal dextrose agar is a selective medium where it deficient of histidine whilst the Pichia host strains GS115 and KM71 have a mutation in the histidinol dehydrogenase gene (his4) which prevents them from synthesizing histidine. All expression plasmids carry the HIS4 gene which complements his4 in the host, so transformants are selected for their ability to grow on histidine-deficient medium (Pichia Expression Kit Manual, Invitrogen, USA). Neither GS115 nor KM71 will grow on minimal medium alone until transformed as they are His-.

d) Screening of Positive Pichia pastoris Transformants

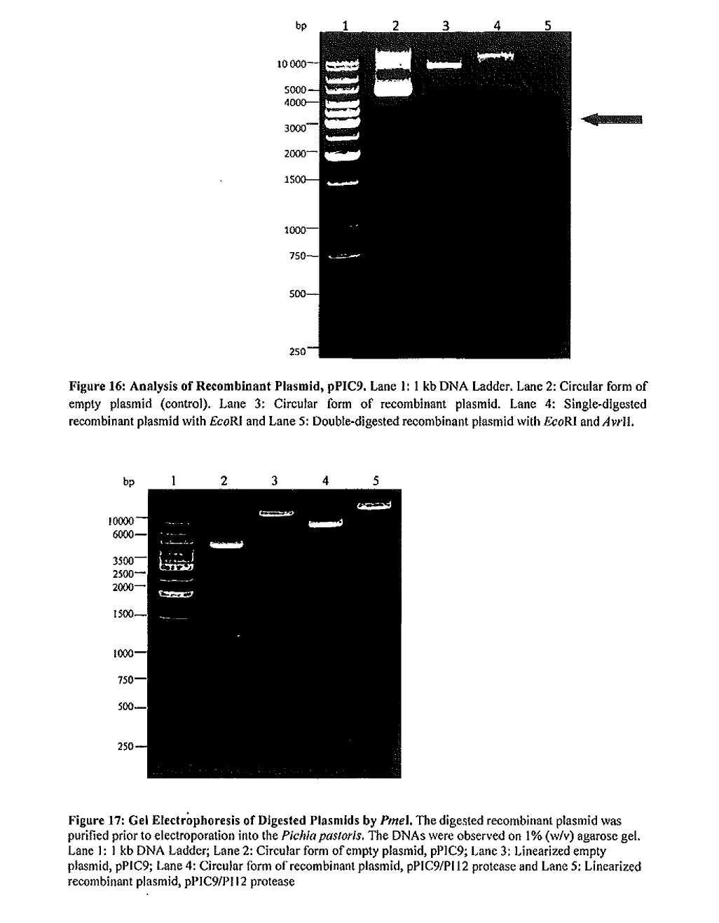

Colonies were picked and resuspended individually in 20 μL of the PCR cocktail consisting of 1.5 mM MgCl₂, 10×PCR buffer containing (NH₄)₂SO₄, 0.2 mM dNTP mix, 2 units Tag DNA polymerase, 10 pmole each reverse and forward primers which were mentioned previously and 50 ng DNA template. The reaction mixture was amplified in a thermocycler (CG1-96 Corbett Research, Australia) for 30 cycles with an annealing temperature of 58° C., steps and condition as described previously. Electrophoresis was carried out through 1% (w/v) agarose gel. The positive clones should give DNA band correspond to size of ˜2.7 kb on the agarose gel. Screening for positive transformants was conducted via PCR which known as a very sensitive and robust method for direct selection of clones for subsequent expression analysis. Besides, no additional cell disruption techniques were required as the heating cycles during the amplification aids in lysis of the yeast cells and at the same time it screens many clones rapidly at once for the present of heterologous sequences (Miles et al., 1998). FIG. 18 shows the PCR products from the positive colonies of recombinant P. pastoris. Only two recombinant clones from GS115 (His⁺ Mut⁺) strain (GpPro1 and GpPro2) and one from KM71 (His⁺ Mut^{S}) strain (KpPro1) were successfully achieved using colony PCR method which were further analyzed for protein expression

Protein Expression in Pichia pastoris

a) Expression of Recombinant pPIC9/Mature PI12 Protease

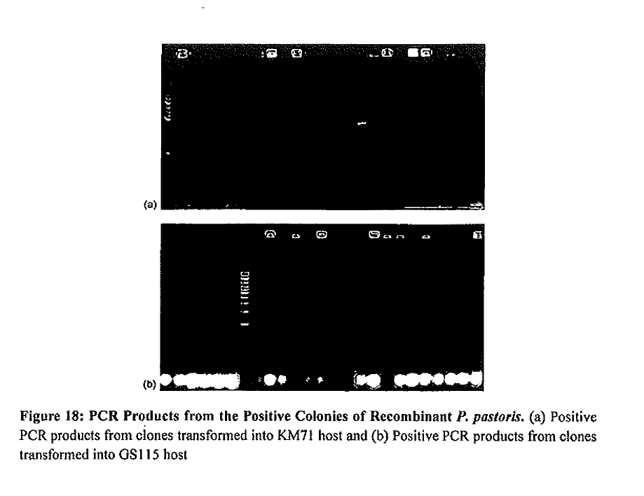

Single colonies of the recombinant Pichia pastoris carrying the pPIC91PI12 protease were grown in 3 mL of BMGY [buffered glycerol-complex medium; 1% (w/v yeast extract, 2% (w/v) peptone, 1.34% (w/v) yeast nitrogen base (YNB), 4×10^{-5%} (w/v) biotin, 1% (w/v) glycerol, 100 mM potassium phosphate buffer, pH 6.0], at 30° C. in a shake incubator (250 rpm) overnight. The next day, 1 mL of the starter cultures were used as inoculum and inoculated into 10 mL of BMGY in a 50 mL flask and incubated in a condition as described to generate cell biomass before induction. Cells were harvested by centrifugation at 1500×g at RT for 10 min. The cells, were resuspended in 50 mL BMMY medium [buffered methanol-complex medium; 1% (w/v) yeast extract, 2% (w/v) peptone, 1.34% (w/v) yeast nitrogen base (YNB), 4×10^{-5%} (w/v) biotin, 0.5% (w/v) methanol, 100 mM potassium phosphate buffer, pH 6.0] with starting optical density OD₆₀₀˜1 and incubate with vigorous shaking at 30° C. for 3 days to induce expression. The expression control was done by applying the same protein expression condition to the recombinant Pichia harbouring the empty vector (pPIC9). After 24 h of incubation, 5 mL of the culture was centrifuged at maximum speed (10,000×g) for 10 min at room temperature. The supernatants were stored at −80° C. until ready to assay. As shown in FIG. 19, the clone with the highest yield is GpPro2 with 20.3 U/mL activity followed by KpPro1 and GpPro1 with 9.1 U/mL and 5.2 U/mL activities, respectively. Therefore, the recombinant PI12 protease was 20.3-fold higher than that produced by its wild-type host (1.0 U/mL). It is apparent that the expression system of Pichia pastoris would seem to be the ideal system for this protein.

b) Assay of Recombinant Protease Activity

Assay activity of recombinant PI12 protease was determined by the modified method of Brock et a (1982) the assay activity for recombinant PI12 protease was performed at 15° C.

c) Detection of Recombinant Protein by SDS-PAGE

The supernatant of recombinant GS115/pPIC9/mature PI12 protease (0.7 mL) was concentrated using 0.7 mL 10% TCA and resuspended in 20 μL phosphate buffer (pH 7.0) and mixed with 5 μL of 5× sample buffer [15 mL 10% (w/v) SDS, 5% (v/v) glycerol, 2.5% (v/v) 2-mercaptoethanol, 6.25% (v/v) 4× upper buffer, 0.005% (w/v) bromophenol blue]. The mixture was then boiled for 10 min. Sodium dodecyl sulphate polyacrylamide gel electrophoresis (SDS-PAGE) was prepared according to the Leammli (1970) method. Table 5 showed the recipes for gels preparation. Electrophoresis was carried out in Tris-Glycine buffer (3% (w/v) tris, 14.4% (w/v) glycine, 0.1% (w/v) SDS; pH 8.4) at constant current of 30-35 mA for 75 min. The gels were then stained with Coomassie Brilliant Blue R-250 [0.5% (w/v) in 25% (v/v) isopropanol and 10% (v/v) acetic acid] for 10 min with gentle agitation at room temperature and de-stained with solution containing 10% (v/v) methanol and 10% (v/v) acetic acid for 1 h.

TABLE 5
Composition for SDS-PAGE

| | Separating gel | Stacking gel |
|---|---|---|
| Components | 15% (w/v) | 5% (w/v) |
| dH₂O | 4.4 mL | 5.9 mL |
| Polyacrylamide mix^{a} | 3.0 mL | 1.5 mL |
| 4x Lower buffer^{b} | 2.5 mL | — |
| 4x Lower buffer^{c} | — | 2.5 mL |
| 10% (v/v) SDS | 0.1 mL | 0.1 mL |
| 10% (w/v) ammonium | 50 μL | 50 μL |
| persulphate | | |
| TEMED | 10 μL | 17 μL |

Note:
^{a}30% (w/v) acrylamide, 0.8% (w/v) bisacrylamide
^{b}1.5M Tris-HCl (pH 8.8)
^{c}0.5M Tris-HCl (pH 6.8)

d) Activity Staining

Activity staining was performed according to Henne et al., 2000. The supernatant from recombinant Pichia clones were applied in this method and loaded into SDS-PAGE [12% (w/v)]. After electrophoresis, gel was immersed in 20% isopropanol to remove SDS and washed with distilled water (2-3 times) and the gel was transferred into 2% skim milk yeast peptone dextrose (YPD) agar plate. The plate was incubated at 15° C. for 5-6 hours. The appearance of a clear zone indicated proteolytic activity due to the hydrolysis of skim milk. The prestained protein molecular weight marker was used to estimate molecular mass.

e) Cup Plate Assay

Protease activity on Petri dishes was tested by the activity ring staining or cup plate assay adapted from Poza et al. (2001) using 2% skim milk yeast peptone dextrose (YPD) agar plate. After solidification, 1.5 mm wells were made, filled with the enzyme solutions and incubated at 15° C. for several hours. 100 μl, of 100% methanol was added to the lid of the inverted plate to compensate for loss due to evaporation or consumption. Protease activity was visualized as clear haloes surrounding the wells.

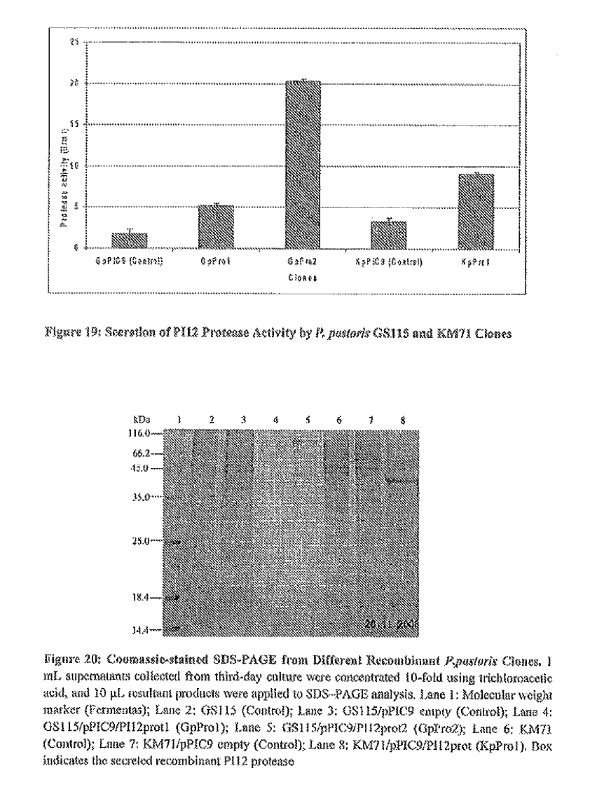

The yeast P. pastoris has proven to be robust and efficient system for expression of PI12 protease. The cold-adapted PI12 protease was effectively expressed in P. pastoris and the recombinant protease was secreted into the expression media. The protease production in P. pastoris was best obtained from strain GS 115 (GpPro2) with 20.3 U/mL activity after 3 days of induction time. The protease assay was carried out under lower temperature, 15° C. to examine for cold-adapted/active property of this enzyme. Interestingly, cold-adapted serine protease from L. antarcticum strain PI12 having the largest molecular mass compared to other cold-adapted protease with predicted molecular mass of 99.3 kDa. FIG. 20 illustrates SDS-PAGE wherein a band at around 99.3 kDa was visibly seen from the recombinant clones' supernatant. GpPro2 colony which contributed the highest protease activity demonstrated the most intense expression band (Lane 5) which proves that the PI12 protease was successfully expressed as fusion protein.

1. An enzyme obtained from a biologically pure strain of Leucosporidium antarcticum sp. isolated from antarctic marine waters.

2. The enzyme as claimed in claim 1, the pure strain having the capability to grow between 4° C. and 20° C.

3. The enzyme of claim 1, wherein the Leucosporidium antarcticum sp or Leucosporidium antarcticum strain PI12 have been deposited in the National Collection of Yeast Cultures (NCYC) under the NCYC number 3391.

4. The enzyme of claim 1, wherein the Leucosporidium antarcticum sp or Leucosporidium antarcticum strain PI12 produces said enzyme.

5. The enzyme as claimed in claim 1, wherein the enzyme is encoded by a nucleotide sequence comprising SEQ ID NO 1
ATGCTCTTCCTCCCCGTCCTCCTCCTCCTCCTTCCCGGCGTCACTGCCTT
CCTCAACCCAGTTACCAACCGCGCGACCAACGCCATCTCCTCGACTCAAT
ACCTCTCTAACGCCTATATCCTCGAATTGGACCTCTCCACCCCCGGCCTC
GTCAAACGGGATAGCACGCCCGACTCTATCCTAGAGGACGTACTCACGTC
CGTAGGCCGCAACGGTATCAAGTACCAACTCCGCCACCGCTTTATCTCCC
CGACTCTGTTCCACGGCGCTTCGATCACTGTCCCCCCTGGAATCTCCCGC
TCCCAAATCGCCTCTCTCCGCGGTATCAAACGCGTCTGGCCCGTTCGAAA
GTTCTCCCGACCCAGCGCAGTAGTGGACGCCGATGGAGGAGGAAGCGGGT
TCTCAGGGTCGCCTATCAAGGCGGCGCTCATGGGGGTGAAAGAGCTCGGG
AAGCGCGCGAACGCTTATGCTGGAGATACGTTTGGACCGCATGTCATGAC
GGGGGTTAATGAGACGCATGAGGCGGGGTTGTTGGGAGCTGGGATTAAGA
TTGGGGTGCTGGACACTGGTGTTGATTATTTGAACCCGATTCTGGGAGGC
TGCTTTGGACCTGGGTGCCATATGTCGTTTGGGTACGACTTGGTTGGCGA
TGATTACGATGGAGATAACGCTCCTGTGCCGGATGTGGATCCTTTTGCGA
GCTGCGATCCTCATGGAACTCACGTTACGGGAATCATTGGAGCGCTCCCG
AATGCGTTTGGATTTACTGGCGTCGCACCCGCCGCTACTCTGGGCCACTA
CCGAGTATTTGGCTGCACTGGCTTCGTCGGAGAAGATATCATTCTCGCTG
GACTCATGCGAGGAGTCGAGGACAACTGCAACGTCTTGACCCTCTCTCTC
GGAGGTCCAGGAGGGTGGGTCAAGGGCACGCCGGCGTCCATCCTTATCGA
CCAGATCGAAGCGCAAGGCATTCTCGTCACCGTCGCCACTGGCAACTCGG
GAGCTGAGGGCATGTTCTTCTCCGAGTCTCCCGCCTCGACGATCAACGGC
CTTTCCATCGCATCCACGGACGTTACCGACCTCATCGCCTACAACGCCAC
CGTCTCAGGCCAACCTGCGATCCCTTACCTCTCCGCCACGCCCCTCAACG
TCGTCGCCAACAGCTTCCGCGTCCACTTCACCTCTACCGACCCCAACAAC
CCCGTCGACGCCTGCTCTCCTCTTCCGGCTGGAGCGCCCGACTTCGCCAA
CTATGTTACGGTCGTTCAGCGTGGGACTTGTACGTTCGTTACCAAGTACC
AGAACGTTCTCAATGCTGGAGGAAAGATCGTATTGTTGTACAACTCGGAG
GGAGCTGGGAACCTCCCTTACCTCACGCCCAACGGTGTCGOCATCGACGC
CGTTGCAGGTCTTCGTCGTTCCGACGGACTCAAGCTTCTCTCGTACTATC
AGAATGCCAACAAGCGTCTCACTCTGCGCTTCCCCAAGGGCAAGATCGTC
GCAGGCTTGACCGATACCATCACCGGCGGACTCATCTCGGGTTACTCGAC
GTTTGGTCCGACGAATGACCTCTACGGTCAGCCTACCCTCTCTGCCCCTG
GTGGCAACATCCTTTCGACCTTCCCTCTCTCCGAGGGAGGAGTGGCGGTC
ATCAGTGGGACGAGCATGTCGTGCCCCTTTGTCGCTGGATCTGCGGCGGT
CCTCATGGCCGCTCGCGCTTCGGAGAACCTCACGCCGCTTGAGATCAGGA
GTCTCCTTACTACCACTGCGAAGCTTACGCCGGTCTCGCTCTTGGGATCG
ACGCCTTTGGTGAGCGTGATTCGTCAAGGAGGAGGACTCGTTCAGGTTGC
CAAGGCGCTCGCGGCCAAGACGCTAATCTCTCCTCACGAGCTCCTGCTCA
ACGACACTGCGAACGCGAACTACGTCCAGACTATCAAGATCAAGAACACC
AACTCGTGGGCGATGAAGTACACCTTCTCCTCGGCCGTCGCCCAAGGACT
CGGAACTTTCGACGCTTCGGGCGATATCCTCCCTACCCTCGACCCAGTCG
CCGTCTCTGGCGCACAGGCTACCGTCGCGTTCAACACTCGGATCCTCAGC
GTCGCGCCCGGCGCGACGGGGTCCGTCGTGGCGACTATCACGCCGCCGGT
TCTTCCCGTAGCGGACGCTGCGAGGTTCCCTATCTTCTCTGGGTGGATCA
GGGTGAATGGGCAAGGCGCGAGGGATAGCAGTAGGAACGAGGCGTACACT
GTCCCGTACTTTGGGCTTGCGGCGAAGATGATCGATATGCAAGTCCTCGA
CACCACCGAGACCATTTACGGTCCGGGCTACGCCTACCCCTTCGTGATCG
ACGACGCGATTGGAGACATCCAATCCACCACAACGTCGTACTCCAGGAAC
CTCGGGCCCACCGTCTTCGCTCGCTTTGCCACTGGAACCCTTCACTACAG
CCTCGATCTCGTCCTAGCCGACATCGCCTTTACCCCCACCTACCCCAACT
CCTCCCCCGCCACTCGTTTCGTCAAGCGCTCCCTCACGCAGCACACCTCT
GCCGCTTCGCACCTCGCCAAGCGCCGCGTCTCCATCGCCACTATCAACCC
CAAAGCCACCCTCGTCGCCGATCGACAGCTCCACTCGGACGTCCCTATCG
AGGGCAACATCTTCACCCAACCCTTTACTGGAAGGGATTACCTCGTCGAC
GCAGCCCCGACGGGATCCACCGATCGTACCGTCACTTTTAACGGGCAGTA
CGCCGAGAACGGCCTCGTGAGGACGGCTGTGACGGGGACTTCGTACCGCT
TCCTCCTTCGGGCGTTGAAGATCTCGGGAGACGCGATGTACGAGGATCAG
TATGAGAGCTGGCTCTCGCTACCGTTCTCGTTCCGTGCGTAG

6. The enzyme as claimed in claim 1, wherein the enzyme comprises an amino acid sequence of SEQ ID NO: 2.
MLFLPVLLLLLPGVTAFLNPVTNRATNAISSTQYLSNAYILELDLSTPGLVKRDSTPDSILEDVLTSVGR
NGIKYQLRHRFISPTLFHGASITVPPGISRSQIASLRGIKRVWPVRKFSRPSAVVDADGGGSGFSGSPIK
AALMGVKELGKRANAYAGDTFGPHVMTGVNETHEAGLLGAGIKIGVLDTGVDYLNPILGGCFGPGCHMSF
GYDLVGDDYDGDNAPVPDVDPFASCDPHGTHVTGIIGALPNAFGFTGVAPAATLGHYRVFGCTGFVGEDI
ILAGLMRGVEDNCNVLTLSLGGPGGWVKGTPASILIDQIEAQGILVTVATGNSGAEGMFFSESPASTING
LSIASTDVTDLIAYNATVSGQPAIPYLSATPLNVVANSFRVHFTSTDPNNPVDACSPLPAGAPDFANYVT
VVQRGTCTFVTKYQNVLNAGGKIVLLYNSEGAGNLPYLTPNGVGIDAVAGLRRSDGLKLLSYYQNANKRL
TLRFPKGKIVAGLTDTITGGLISGYSTFGPTNDLYGQPTLSAPGGNILSTFPLSEGGVAVISGTSMSCPF
VAGSAAVLMAARASENLTPLEIRSLLTTTAKLTPVSLLGSTPLVSVIRQGGGLVQVAKALAAKTLISPHE
LLLNDTANANYVQTIKIKNTNSWAMKYTFSSAVAQCLGTFDRSGDILPTLDPVAVSGAQATVAFNTRILS
VAPGATGSVVATITPPVLPVADAARFPIFSGWIRVNGQGARDSSRNEAYTVPYFGLAAKMIDMQVLDTTE
TIYGPGYAYPFVIDDAIGDIQSTTTSYSRNLGPTVFARFATGTLHYSLDLVLADIAFTPTYPNSSPATRF
VKRSLTQHTSAASHLAKRRVSIATINPKATLVADRQLHSDVPIEGNIFTQPFTGRDYLVDAAPTGSTDRT
VTFNGQYAENGLVRTAVTGTSYRFLLRALKISGDAMYEDQYESWLSLPFSFRA

7. The enzyme as claimed in claim 1, wherein the enzyme is a protease.

8. The enzyme as claimed in claim 1, wherein the protease is a cold active protease PI12.

9. A method of indentifying the Leucosporidium antarcticum sp, wherein the method comprising the steps of:
a) isolating the Leucosporidium antarcticum PI12 in a solid media and antibiotics for at least 10 days at 4° C. and characterizing the Leucosporidium antarcticum PI12 as psychrophilic isolate PI12,
b) identifying the morphology and size of the psychrophilic isolate,
c) scanning the psychrophilic isolate under an electron microscopy and transmission microscopy,
d) conducting a ribosomal RNA identification by using 16srRNA, 18rRNA and ITS1/ITS2 primers,
e) amplifying the primers from step (d) by performing PCR,
f) obtaining amplicons from step (e) and examining the amplicons,
g) purifying and sequencing the amplicons and obtaining sequences from the above primers.

10. The method as claimed in claim 9, wherein the Leucosporidium antarcticum sp is resistant to ampicillin, streptomycin and chloramphenicol.

11. The method as claimed in claim 9, wherein the Leucosporidium antarcticum sp comprising a nucleotide sequence of SEQ ID NO 3 and SEQ ID NO 4.

12. A gene coding a protein from the cold active protease PI12 enzyme of claim 8.

13. The gene as claimed in claim 12, wherein the protein has a size of about 99.3 kDa.

14. A method of obtaining a PI12 protease gene isolated from Leucosporidium antarcticum sp, wherein the method comprising the steps of:
a) conducting DNA extraction of the Leucosporidium antarctic=sp,
b) obtaining a purified DNA extract from step (a),
c) identifying the partial putative protease gene in a recombinant plasmid by performing double digestion using EcoRI restriction enzyme at 37° C. for 1 hour and terminated at 65° C. for 20 minutes,
d) obtaining a digested product from step (c) and sequencing the digested product,
e) conducting RNA extraction of the Leucosporidium antarcticum sp.
f) performing RT-PCR to amplify the protease gene,
g) performing an amplification of cDNA ends and obtaining PCR product,
h) cloning and sequencing the PCR product from step (g),
i) obtaining a full length sequence of a mature PI12 protease gene.

15. The method as claimed in claim 14, wherein the PI12 protease gene is amplified at 2892 bp and encodes for 963 amino acids.

16. The method as claimed in claim 14, wherein the method further includes the step of:
a) cloning the mature PI12 protease gene into an expression vector,
b) obtaining low temperature or cold-adapted PI12 protease clones from step (a) at 15° C. for about 30 minutes.

17. The method as claimed in claim 14 (a), wherein the expression vector is Pichia pastoris (pPIC9).

18. The method as claimed in claim 16 (b), the clones includes GS115 strain and KM71 strain, wherein GS115 strain is GpPro1 and GpPro2 and the KM71 strain is KpPro1.

19. The method as claimed in claim 16 (b), wherein the clones were further verified by assaying with azocasein as a substrate and terminated using trichloroacetic acid.


SEQUENCE LISTING (44 sequences)

SEQ ID NO: 1 - DNA, 2892 bp, Leucosporidium antarcticum
atgctcttcctccccgtcctcctcctcctccttcccggcgtcactgccttcctcaacccagttaccaaccgcgcgaccaacgccatctcctcgactcaatacctctctaacgcctatatcctcgaattggacctctccacccccggcctcgtcaaacgggatagcacgcccgactctatcctagaggacgtactcacgtccgtaggccgcaacggtatcaagtaccaactccgccaccgctttatctccccgactctgttccacggcgcttcgatcactgtcccccctggaatctcccgctcccaaatcgcctctctccgcggtatcaaacgcgtctggcccgttcgaaagttctcccgacccagcgcagtagtggacgccgatggaggaggaagcgggttctcagggtcgcctatcaaggcggcgctcatgggggtgaaagagctcgggaagcgcgcgaacgcttatgctggagatacgtttggaccgcatgtcatgacgggggttaatgagacgcatgaggcggggttgttgggagctgggattaagattggggtgctggacactggtgttgattatttgaacccgattctgggaggctgctttggacctgggtgccatatgtcgtttgggtacgacttggttggcgatgattacgatggagataacgctcctgtgccggatgtggatccttttgcgagctgcgatcctcatggaactcacgttacgggaatcattggagcgctcccgaatgcgtttggatttactggcgtcgcacccgccgctactctgggccactaccgagtatttggctgcactggcttcgtcggagaagatatcattctcgctggactcatgcgaggagtcgaggacaactgcaacgtcttgaccctctctctcggaggtccaggagggtgggtcaagggcacgccggcgtccatccttatcgaccagatcgaagcgcaaggcattctcgtcaccgtcgccactggcaactcgggagctgagggcatgttcttctccgagtctcccgcctcgacgatcaacggcctttccatcgcatccacggacgttaccgacctcatcgcctacaacgccaccgtctcaggccaacctgcgatcccttacctctccgccacgcccctcaacgtcgtcgccaacagcttccgcgtccacttcacctctaccgaccccaacaaccccgtcgacgcctgctctcctcttccggctggagcgcccgacttcgccaactatgttacggtcgttcagcgtgggacttgtacgttcgttaccaagtaccagaacgttctcaatgctggaggaaagatcgtattgttgtacaactcggagggagctgggaacctcccttacctcacgcccaacggtgtcggcatcgacgccgttgcaggtcttcgtcgttccgacggactcaagcttctctcgtactatcagaatgccaacaagcgtctcactctgcgcttccccaagggcaagatcgtcgcaggcttgaccgataccatcaccggcggactcatctcgggttactcgacgtttggtccgacgaatgacctctacggtcagcctaccctctctgcccctggtggcaacatcctttcgaccttccctctctccgagggaggagtggcggtcatcagtgggacgagcatgtcgtgcccctttgtcgctggatctgcggcggtcctcatggccgctcgcgcttcggagaacctcacgccgcttgagatcaggagtctccttactaccactgcgaagcttacgccggtctcgctcttgggatcgacgcctttggtgagcgtgattcgtcaaggaggaggactcgttcaggttgccaaggcgctcgcggccaagacgctaatctctcctcacgagctcctgctcaacgacactgcgaacgcgaactacgtccagactatcaagatcaagaacaccaactcgtgggcgatgaagtacaccttctcctcggccgtcgcccaaggactcggaactttcgacgcttcgggcgatatcctccctaccctcgacccagtcgccgtctctggcgcacaggctaccgtcgcgttcaacactcggatcctcagcgtcgcgcccggcgcgacggggtccgtcgtggcgactatcacgccgccggttcttcccgtagcggacgctgcgaggttccctatcttctctgggtggatcagggtgaatgggcaaggcgcgagggatagcagtaggaacgaggcgtacactgtcccgtactttgggcttgcggcgaagatgatcgatatgcaagtcctcgacaccaccgagaccatttacggtccgggctacgcctaccccttcgtgatcgacgacgcgattggagacatccaatccaccacaacgtcgtactccaggaacctcgggcccaccgtcttcgctcgctttgccactggaacccttcactacagcctcgatctcgtcctagccgacatcgcctttacccccacctaccccaactcctcccccgccactcgtttcgtcaagcgctccctcacgcagcacacctctgccgcttcgcacctcgccaagcgccgcgtctccatcgccactatcaaccccaaagccaccctcgtcgccgatcgacagctccactcggacgtccctatcgagggcaacatcttcacccaaccctttactggaagggattacctcgtcgacgcagccccgacgggatccaccgatcgtaccgtcacttttaacgggcagtacgccgagaacggcctcgtgaggacggctgtgacggggacttcgtaccgcttcctccttcgggcgttgaagatctcgggagacgcgatgtacgaggatcagtatgagagctggctctcgctaccgttctcgttccgtgcgtag

SEQ ID NO: 2 - protein, 963 aa, Leucosporidium antarcticum
MLFLPVLLLLLPGVTAFLNPVTNRATNAISSTQYLSNAYILELDLSTPGLVKRDSTPDSILEDVLTSVGRNGIKYQLRHRFISPTLFHGASITVPPGISRSQIASLRGIKRVWPVRKFSRPSAVVDADGGGSGFSGSPIKAALMGVKELGKRANAYAGDTFGPHVMTGVNETHEAGLLGAGIKIGVLDTGVDYLNPILGGCFGPGCHMSFGYDLVGDDYDGDNAPVPDVDPFASCDPHGTHVTGIIGALPNAFGFTGVAPAATLGHYRVFGCTGFVGEDIILAGLMRGVEDNCNVLTLSLGGPGGWVKGTPASILIDQIEAQGILVTVATGNSGAEGMFFSESPASTINGLSIASTDVTDLIAYNATVSGQPAIPYLSATPLNVVANSFRVHFTSTDPNNPVDACSPLPAGAPDFANYVTVVQRGTCTFVTKYQNVLNAGGKIVLLYNSEGAGNLPYLTPNGVGIDAVAGLRRSDGLKLLSYYQNANKRLTLRFPKGKIVAGLTDTITGGLISGYSTFGPTNDLYGQPTLSAPGGNILSTFPLSEGGVAVISGTSMSCPFVAGSAAVLMAARASENLTPLEIRSLLTTTAKLTPVSLLGSTPLVSVIRQGGGLVQVAKALAAKTLISPHELLLNDTANANYVQTIKIKNTNSWAMKYTFSSAVAQGLGTFDASGDILPTLDPVAVSGAQATVAFNTRILSVAPGATGSVVATITPPVLPVADAARFPIFSGWIRVNGQGARDSSRNEAYTVPYFGLAAKMIDMQVLDTTETIYGPGYAYPFVIDDAIGDIQSTTTSYSRNLGPTVFARFATGTLHYSLDLVLADIAFTPTYPNSSPATRFVKRSLTQHTSAASHLAKRRVSIATINPKATLVADRQLHSDVPIEGNIFTQPFTGRDYLVDAAPTGSTDRTVTFNGQYAENGLVRTAVTGTSYRFLLRALKISGDAMYEDQYESWLSLPFSFRA

SEQ ID NO: 3 - DNA, 1739 bp, Leucosporidium antarcticum
gcttgtctcaagattaagccatgcatgtctaagtttaagcaataaacggtgaaactgcgaatggctcattaaatcagtcatagtttatttgatggtaccctactacatggataactgtggtaattctagagctaatacatgccgaaaaatctcgacttctggaagagatgtatttattagatccaaaaccagtggccttcgggtctccttggtgaatcatgataactgctcgaatcgcatggccttgcgccggcgatgcttcattcaaatatctgccctatcaactttcgatggtaggatagaggcctaccatggtgatgacgggtaacggggaataagggttcgattccggagagagggcctgagaaacggccctcaggtctaaggacacgcagcaggcgcgcaaattatcccctggcaacactttgccgagatagtgacaataaataacaatgcagggctcttacgggtcttgcaattggaatgagtacaatttaaatcccttaacgaggatccattggagggcaagtctggtgccagcagccgcggtaattccagctccaatagcgtatattaaagttgttgccgttaaaaagctcgtagtcgaacttcggtccttgttggtcggtccgccttcttggtgtgtacttactcaacgaggacttacctcctggtgagctgcaatgtcctttactgggtgttgtagggaaccaggacgtttactttgaaaaattagagtgttcaaagcaggcctacgcccgaatacattagcatggaatatagaataggacgcgcgttcccattttgttggtttctgagatcgccgtaatgattaatagggatagttgggggcattcgtattccgtcgtcagaggtgaaattcttggattgccggaagacgaactattgcgaaagcatttgccaaggatgttttcattgatcaagaacgaaggaagggggatcgaaaacgatcagataccgttgttgtctcttctgtaaactatgccaattggggattagctcaggatttttaatgactgagttagcacccgaagagaaatctttaaatgaggttcgggggggagtatggtcgcaaggctgaaacttaaaggaattgacggaagggcaccaccaggtgtggagcctgcggcttaatttgactcaacacggggaaactcaccaggtccagacacaataaggattgacagattgatagctctttcttgatcttgtggttggtggtgcatggccgttcttagttggtggagtgatttgtctggttaattccgataacgaacgagaccttaacctgctaaatagaccagccggctttggctggctgctgtcttcttagagggactatcagcgtttagctgatggaagtttgaggcaataacaggtctgtgatgcccttagatgttctgggccgcacgcgcgctacactgacagagccagcgagtctaccaccttggccggaaggcctgggtaatcttgtgaaactctgtcgtgatggggatagaacattgcaattattgttcttcaacgaggaatacctagtaagcgtgagtcatcagctcgcgttgattacgtccctgccctttgtacacaccgcccgtcgctactaccgattgaatggcttagtgaggcctccagattggctattaggatctcgcgagagaacttgactgctgaaaagttgtacgaacttggtcatttagaggaagtaaaagtcgtaacaa

SEQ ID NO: 4 - DNA, 608 bp, Leucosporidium antarcticum
tggggaaggatcattagcgaatttagcgtttctcttaacagagcgcgaccctccactttcttaactctgtgaacttttttggtcaagcatggcgttttctcgattgacttttattagaaagttgggactacacattttgcttgacggctcattttaaacactagtacaagtatgtaacgaaatatcgaaatataaaaaaactttcaacaacggatctcttggctctcgcatcgatgaagaacgcagcgaaatgtgataagtaatgtgaattgcagaattcagtgaatcatcgaatctttgaacgcaccttgcgctccctagtattctggggagcatgtctgtttgagtgtcatgaactcttcaactctatcgtttcttgttaagcgattagaagtttggattttgaatgttgctagtccttttacgggactttagctcgttcgtaatacattagcctttctaattccgaacttcggattgactcagtgtaatagactattcgctgaggacactagtaatagtggccgatattcaacataagaaaagcttcaaacctttgtagtcaattttagattagacctcagatcaggcaggattaacccgccgaa

SEQ ID NO: 5 - DNA, 30 bp, Artificial Sequence
  Description of Artificial Sequence Synthetic primer
ccgaattcgtcgacaacagagtttgatcct

SEQ ID NO: 6 - DNA, 30 bp, Artificial Sequence
  Description of Artificial Sequence Synthetic primer
cccgggatccaagcttacggctaccttgtt

SEQ ID NO: 7 - DNA, 21 bp, Artificial Sequence
  Description of Artificial Sequence Synthetic primer
aacctggttgatcctgccagt

SEQ ID NO: 8 - DNA, 24 bp, Artificial Sequence
  Description of Artificial Sequence Synthetic primer
tgatccttctgcaggttcacctac

SEQ ID NO: 9 - DNA, 30 bp, Artificial Sequence
  Description of Artificial Sequence Synthetic primer
tccgtaggtgaacctgcggaaggatcatta

SEQ ID NO: 10 - DNA, 30 bp, Artificial Sequence
  Description of Artificial Sequence Synthetic primer
cttttcctccgcttcttgatatgcttaagt

SEQ ID NO: 11 - DNA, 21 bp, Artificial Sequence
  Description of Artificial Sequence Synthetic primer
tcacagaagtatgccaagcga

SEQ ID NO: 12 - DNA, 19 bp, Artificial Sequence
  Description of Artificial Sequence Synthetic primer
agggtcaagacgttgcagt

SEQ ID NO: 13 - DNA, 22 bp, Artificial Sequence
  Description of Artificial Sequence Synthetic primer
atgcgaagtcagaagcaggatc

SEQ ID NO: 14 - DNA, 22 bp, Artificial Sequence
  Description of Artificial Sequence Synthetic primer
gcagccaaatacctggaagcac

SEQ ID NO: 15 - DNA, 20 bp, Artificial Sequence
  Description of Artificial Sequence Synthetic primer
tttttttttttttttttttt

SEQ ID NO: 16 - DNA, 26 bp, Artificial Sequence
  Description of Artificial Sequence Synthetic primer
atgctcttcctccccgtcctcctcct

SEQ ID NO: 17 - DNA, 24 bp, Artificial Sequence
  Description of Artificial Sequence Synthetic primer
tcagcgaacgaacgaggagaaggt

SEQ ID NO: 18 - DNA, 30 bp, Artificial Sequence
  Description of Artificial Sequence Synthetic primer
aagcagtggtatcaacgcagagtacgcggg

SEQ ID NO: 19 - DNA, 27 bp, Artificial Sequence
  Description of Artificial Sequence Synthetic primer
tttttttttttttttttttttttttvn

SEQ ID NO: 20 - DNA, 57 bp, Artificial Sequence
  Description of Artificial Sequence Synthetic primer
aagcagtggtatcaacgcagagtacttttttttttttttttttttttttttttttvn

SEQ ID NO: 21 - DNA, 22 bp, Artificial Sequence
  Description of Artificial Sequence Synthetic primer
ctaatacgactcactatagggc

SEQ ID NO: 22 - DNA, 29 bp, Artificial Sequence
  Description of Artificial Sequence Synthetic primer
accagtgtccagcaccccaatcttaatcc

SEQ ID NO: 23 - DNA, 24 bp, Artificial Sequence
  Description of Artificial Sequence Synthetic primer
tcatcagtgggacgagcatgtcgt

SEQ ID NO: 24 - DNA, 21 bp, Artificial Sequence
  Description of Artificial Sequence Synthetic primer
gcgggggccgacaataaaaac

SEQ ID NO: 25 - DNA, 34 bp, Artificial Sequence
  Description of Artificial Sequence Synthetic primer
ttttttttttttttttttttttttttgaggtggc

SEQ ID NO: 26 - DNA, 29 bp, Artificial Sequence
  Description of Artificial Sequence Synthetic primer
caagccctaggctacgcacggaacgagaa

SEQ ID NO: 27 - DNA, 32 bp, Artificial Sequence
  Description of Artificial Sequence Synthetic primer
ctgccgaattcttcctcaacccagttaccaac

SEQ ID NO: 28 - DNA, 21 bp, Artificial Sequence
  Description of Artificial Sequence Synthetic primer
tactattgccagcattgctgc

SEQ ID NO: 29 - DNA, 21 bp, Artificial Sequence
  Description of Artificial Sequence Synthetic primer
gactggttccaattgacaagc

SEQ ID NO: 30 - DNA, 21 bp, Artificial Sequence
  Description of Artificial Sequence Synthetic primer
gcaaatggcattctgacatcc

SEQ ID NO: 31 - DNA, 1321 bp, Leucosporidium antarcticum
cgcagctcccactcactcgtgcttccaggtatttggctgcactggcttcgtcggagaagatatcgttcgtcctctctccagtcccggctagcgatcctgcttctgacttcgcatctccctctcagattctcgctggactcatgcgaggagtcgaggacaactgcaacgtcttgaccctctctctcggaggtccaggagggtgggtcaagggcacgccggcgtccatccttatcgaccagatcgaagcgcaaggcattctcgtcaccgtcgccactggcaactcgggagctgagggtgagctcctcgctacttgcgcgtcgagaccagtactaacaggggcgtggacgcaggcatgttcttctccgagtctcccgcctcgacgatcaacggcctttccatcgcatccacgtccgtctctcccttccttcgcacgccactcgctcacactctctcgcagggacgttaccgacctcatcgcctacaacgccaccgtctcaggccaacctgcgatcccttacctctccgccacgcccctcaacgtcgtcgccaacagcttccgcgtccacttcacctctaccgaccccaacaaccccgtcgacgcctgctctcctcttccggctggaacgcccgacctcgccaactatgttacggtcgttcagcgtgggacttgtacgttcgttaccaagtaccagaacgttctcaatgctggagggtgcgtcggctcttctctcccgactggattgggcggagactgaccttctgagcagaaagatcgtattgttgtacaactcggagggagctgggaacctcccttacctcacgcccaacggtgtcggcatcgacgccgttgcaggtcttcgtcgttccgacggactcaaggttcgtctacgctcttcggtgctgaggcccttctctgatctcgctccgacagcttctctcgtactatcagaatgccaacaagcgtctcactctgcgcttccccaagggcaagatcgtcgcaggcttgaccgacaccatcaccggcggactcatctcgttcgttcctctccgcctccctcgctggccgatgagctgacgcggtctttgcaggagttactcgacgtttggtccgacgaatgacctctacggtcagcctaccctctctgcccctggtggcaacatcctttcgaccttccctctctccgagggaggagtggcggtcatcagtgggacgagcatgtcgtgcccctttgtcggttcgtccttctcctcgttcgttcgctgagcgtcgactgatcttgcacgatcgcagctggatctgcggcggtcctcatggccgctc

SEQ ID NO: 32 - DNA, 177 bp, Leucosporidium antarcticum
atgctcttcctccccgtcctcctcctcctccttcccggcgtcactgccttcctcaacccagttaccaaccgcgcgaccaacgccatctcctcgactcaatacctctctaacgcctatatcctcgaattggacctctccacccccggcctcgtcaaacgggatagcacgcccgactct

SEQ ID NO: 33 - DNA, 153 bp, Leucosporidium antarcticum
atcctagaggacgtactcacgtccgtaggccgcaacggtatcaagtaccaactccgccaccgctttatctccccgactctgttccacggcgcttcgatcactgtcccccctggaatctcccgctcccaaatcgcctctctccgcggtatcaaa

SEQ ID NO: 34 - DNA, 184 bp, Leucosporidium antarcticum
cgcagtagtggacgccgatggaggaggagcgggttctcagggtcgcctatcaaggcggcgctcatgggggtgaaagagctcgggaagcgcgcgaacgcttatgctggagatacgtttggaccgcatgtcatgacgggggttaatgagacgcatgaggcggggttgttgggagctgggattaagg

SEQ ID NO: 35 - DNA, 95 bp, Leucosporidium antarcticum
ctgctttggacctgggtgccatatgtcgtttgggtacgacttggttggcgatgattacgatggagataacgctcctgtgccggatgtggatcctt

SEQ ID NO: 36 - DNA, 22 bp, Leucosporidium antarcticum
ttgcgagctgcgatcctcatgg

SEQ ID NO: 37 - DNA, 90 bp, Leucosporidium antarcticum
aactcacgttacgggaatcattagagcgctcccgaatgcgtttggatttactggcgtcgcacccgccgctactctgggccactaccgagt

SEQ ID NO: 38 - DNA, 265 bp, Leucosporidium antarcticum
atttggctgcactggcttcgtcggagaagatatcgttcgtcctctctccagtcccggctagcgatcctgcttctgacttcgcatctccctctcagattctcgctggactcatgcgaggagtcgaggacaactgcaacgtcttgaccctctctctcggaggtccaggagggtgggtcaagggcacgccggcgtccatccttatcgaccagatcgaagcgcaaggcattctcgtcaccgtcgccactggcaactcgggagctgaggg

SEQ ID NO: 39 - DNA, 58 bp, Leucosporidium antarcticum
catgttcttctccgagtctcccgcctcgacgatcaacggcctttccatcgcatccacg

SEQ ID NO: 40 - DNA, 254 bp, Leucosporidium antarcticum
gacgttaccgacctcatcgcctacaacgccaccgtctcaggccaacctgcgatcccttacctctccgccacgcccctcaacgtcgtcgccaacagcttccgcgtccacttcacctctaccgaccccaacaaccccgtcgacgcctgctctcctcttccggctggaacgcccgacctcgccaactatgttacggtcgttcagcgtgggacttgtacgttcgttaccaagtaccagaacgttctcaatgctggagg

SEQ ID NO: 41 - DNA, 112 bp, Leucosporidium antarcticum
aaagatcgtattgttgtacaactcggagggagctgggaacctcccttacctcacgcccaacggtgtcggcatcgacgccgttgcaggtcttcgtcgttccgacggactcaag

SEQ ID NO: 42 - DNA, 105 bp, Leucosporidium antarcticum
cttctctcgtactatcagaatgccaacaagcgtctcactctgcgcttccccaagggcaagatcgtcgcaggcttgaccgacaccatcaccggcggactcatctcg

SEQ ID NO: 43 - DNA, 174 bp, Leucosporidium antarcticum
agttactcgacgtttggtccgacgaatgacctctacggtcagcctaccctctctgcccctggtggcaacatcctttcgaccttccctctctccgagggaggagtggcggtcatcagtgggacgagcatgtcgtgcccctttgtcggttcgtccttctcctcgttcgttcgctga

SEQ ID NO: 44 - DNA, 2687 bp, Leucosporidium antarcticum
tcacagaagtatgccaagcgaggggggggggtctgctagcgcgaatcgagaaaacacaaaagaacctcccgacttggcgttgcttctgctcttctccttctcccaaccgccgacaataaaaactataacacacacacaccccatctcgacgcctgcagacttcacgactctcctcacgctctttaccgccccacgggtcccacccacgagccacgatgctcttcctccccgtcctcctcctcctccttcccggcgtcactgccttcctcaacccagttaccaaccgcgcgaccaacgccatctcctcgactcaatacctctctaacgcctatatcctcgaattggacctctccacccccggcctcgtcaaacgggatagcacgcccgactctgtgcgtcccgcgttcctccctcgctgtcgaagacgagctaacaatgacgtggtagatcctagaggacgtactcacgtccgtaggccgcaacggtatcaagtaccaactccgccaccgctttatctccccgactctgttccacggcgcttcgatcactgtcccccctggaatctcccgctcccaaatcgcctctctccgcggtatcaaagtccgtccctctctctcccctactccctccgtgctaaccctgcacgcaacagcgcgtctggcccgttcgaagttctcccgacccagcgcagtagtggacgccgatggaggaggagcgggttctcagggtcgcctatcaaggcggcgctcatgggggtgaaagagctcgggaagcgcgcgaacgcttatgctggagatacgtttggaccgcatgtcatgacgggggttaatgagacgcatgaggcggggttgttgggagctgggattaaggtgtgttttcgtctttgtttgggggaggggggagggaggagctgattaatgggtgcagattggggtgctggacactggtgagaagcggtttggagggaggagaggatggagctgatgagtgtgcaggtgttgattatttgaacccgattctgggaggctgctttggacctgggtgccatatgtcgtttgggtacgacttggttggcgatgattacgatggagataacgctcctgtgccggatgtggatccttgtgcgtccttccctccgcgatgggctggaatctgagctgacggcgttgttgatcagttgcgagctgcgatcctcatggttcgtctcgctttccgctcctctgatgcgctcgctgatgcacttggcaataggaactcacgttacgggaatcattagagcgctcccgaatgcgtttggatttactggcgtcgcacccgccgctactctgggccactaccgagtgcgctccctctatccgttcttcctcgcagctcccactcactcgtgcttccaggtatttggctgcactggcttcgtcggagaagatatcgttcgtcctctctccagtcccggctagcgatcctgcttctgacttcgcatctccctctcagattctcgctggactcatgcgaggagtcgaggacaactgcaacgtcttgaccctctctctcggaggtccaggagggtgggtcaagggcacgccggcgtccatccttatcgaccagatcgaagcgcaaggcattctcgtcaccgtcgccactggcaactcgggagctgagggtgagctcctcgctacttgcgcgtcgagaccagtactaacaggggcgtggacgcaggcatgttcttctccgagtctcccgcctcgacgatcaacggcctttccatcgcatccacgtccgtctctcccttccttcgcacgccactcgctcacactctctcgcagggacgttaccgacctcatcgcctacaacgccaccgtctcaggccaacctgcgatcccttacctctccgccacgcccctcaacgtcgtcgccaacagcttccgcgtccacttcacctctaccgaccccaacaaccccgtcgacgcctgctctcctcttccggctggaacgcccgacctcgccaactatgttacggtcgttcagcgtgggacttgtacgttcgttaccaagtaccagaacgttctcaatgctggagggtgcgtcggctcttctctcccgactggattgggcggagactgaccttctgagcagaaagatcgtattgttgtacaactcggagggagctgggaacctcccttacctcacgcccaacggtgtcggcatcgacgccgttgcaggtcttcgtcgttccgacggactcaaggttcgtctacgctcttcggtgctgaggcccttctctgatctcgctccgacagcttctctcgtactatcagaatgccaacaagcgtctcactctgcgcttccccaagggcaagatcgtcgcaggcttgaccgacaccatcaccggcggactcatctcgttcgttcctctccgcctccctcgctggccgatgagctgacgcggtctttgcaggagttactcgacgtttggtccgacgaatgacctctacggtcagcctaccctctctgcccctggtggcaacatcctttcgaccttccctctctccgagggaggagtggcggtcatcagtgggacgagcatgtcgtgcccctttgtcggttcgtccttctcctcgttcgttcgctgagcgtcgactgatcttgcacgatcgcagctggatctgcggcggtcctcatggccgctc